(19) United States
(12) Patent Application Publication
(10) Pub. No.: US 2009/0062602 A1
(43) Pub. Date: Mar. 5, 2009

(54) APPARATUS FOR ROBOTIC INSTRUMENT HAVING VARIABLE FLEXIBILITY AND TORQUE TRANSMISSION

(75) Inventors: Craig R. Rosenberg, Palo Alto, CA (US); Jeffrey B. Alvarez, Redwood City, CA (US); Frederic H. Moll, San Francisco, CA (US); Daniel T. Wallace, Burlingame, CA (US); Jason K. Chan, Fremont, CA (US)

Correspondence Address: VISTA IP LAW GROUP LLP, 12930 Saratoga Avenue, Suite D-2, Saratoga, CA, 95070, US

(73) Assignee: Hansen Medical, Inc., Mountain View, CA (US)

(21) Appl. No.: 12/182,980

(22) Filed: Jul. 30, 2008

Related U.S. Application Data
(60) Provisional application No. 60/962,704, filed on Jul. 30, 2007.

(51) Int. Cl. A61B 1/00; A61M 25/00

(52) U.S. Cl. 600/101; 600/585

(57) ABSTRACT

A flexible spine for use in one or more surgical instruments including a catheter and/or sheath of a robotic instrument system. The spine includes an elongate body that defines a central lumen and that is a unitary structure having a plurality of discrete sections, each of which has a distinguishing structural attribute that differentiates it from the other sections. Such distinguishing structural attributes may include, without limitation, materials, material attributes, shapes, sizes and/or attributes related to apertures in a wall of the elongate body, such as a number, shape, size, spacing and degree of overlap of such apertures. The arrangement of discrete, structurally different sections results in varying flexibility of the elongate spine and of corresponding sections of a surgical instrument incorporating the spine.

### CROSS-REFERENCE TO RELATED APPLICATION

The present application claims the benefit under 35 U.S.C. §119 to U.S. Provisional Application No. 60/962,704, entitled “Robotic Instrument and Assemblies” filed on Jul. 30, 2007, the contents of which are incorporated herein by reference as though set forth in full.

The present application may also be related to subject matter disclosed in the following applications, the contents of which are also incorporated herein by reference as though set forth in full: U.S. patent application Ser. No. 11/481,433, entitled “Robotic Catheter System and Methods”, filed Jul. 3, 2006; U.S. patent application Ser. No. 11/637,951, entitled “Robotic Catheter System and Methods”, filed Dec. 11, 2006; and U.S. patent application Ser. No. 12/032,626, entitled “Instrument Assembly for Robotic Instrument System”, filed Feb. 15, 2008.

### FIELD OF INVENTION

The invention relates generally to robotically controlled systems such as telerobotic surgical systems, and more particularly, to flexible and torquable devices and instruments for use in telerobotic surgical systems.

### BACKGROUND

Robotic interventional systems and devices are well suited for use in performing minimally invasive medical procedures as opposed to conventional procedures that involve opening the patient's body to permit the surgeon's hands to access internal organs. Traditionally, surgery utilizing conventional procedures meant significant pain, long recovery times, lengthy work absences, and visible scarring. Advances in surgical technologies have resulted increased use of less invasive surgical procedures, in particular, minimally invasive surgery (MIS). A “minimally invasive medical procedure” is generally considered a procedure that is performed by entering the body through the skin, a body cavity, or an anatomical opening utilizing small incisions rather than larger, more invasive open incisions that are used in various known procedures.

Various medical procedures are considered to be minimally invasive and may involve minor and more complex procedures. Examples of MIS procedures include mitral and tricuspid valve procedures, patent formen ovale, atrial septal defect surgery, colon and rectal surgery, laparoscopic appendectomy, laparoscopic esophagectomy, laparoscopic hysterectomies, carotid angioplasty, vertebroplasty, endoscopic sinus surgery, thoracic surgery, donor nephrectomy, hypodermic injection, air-pressure injection, subdermal implants, endoscopy, percutaneous surgery, laparoscopic surgery, arthroscopic surgery, cryosurgery, microsurgery, biopsies, videoscope procedures, keyhole surgery, endovascular surgery, coronary catheterization, permanent spinal and brain electrodes, stereotactic surgery, and radioactivity-based medical imaging methods. With MIS, it is possible to achieve less operative trauma for the patient, reduced hospitalization time, less pain and scarring, reduced incidence of complications related to surgical trauma, lower costs, and a speedier recovery. Such procedures may involve robotic and computer technologies, and the integration of robotic technologies with surgeon skill into surgical robotics enables surgeons to perform surgical procedures in new and more effective ways.

Although MIS techniques have advanced, physical limitations of certain types of medical equipment can be improved. For example, during a MIS procedure, catheters (e.g., a sheath catheter, a guide catheter, an ablation catheter, etc.) and endoscopes or laparoscopes may be inserted into a body cavity duct or vessel. A catheter is an elongated tube that may, for example, allow for drainage or injection of fluids or provide a path for delivery of working or surgical instruments to a target site. One MIS procedure involves advancing one or more catheters and other surgical instruments through an incision at the femoral vein near the thigh or pelvic region of the patient, which is at some distance away from the operation or target site. In this example, the operation or target site for performing cardiac ablation is in the left atrium of the heart. Catheters are guided (e.g., by a guide wire, etc.) manipulated, and advanced toward the target site by way of the femoral vein to the inferior vena cava into the right atrium through the interatrial septum to the left atrium of the heart. Catheters may be used to apply cardiac ablation therapy to the left atrium of the heart to restore normal heart function to treat cardiac arrhythmias such as atrial fibrillation.

In known robotic instrument systems, however, the ability to control and manipulate system components such as catheters and associated working instruments may be limited due, in part, to a surgeon not having direct access to the target site and not being able to directly handle or control the working instrument at the target site. More particularly, MIS diagnostic and interventional operations require the surgeon to remotely approach and address the operation or target site by using instruments that are guided, manipulated and advanced through a natural body orifice such as a blood vessel, esophagus, trachea, small intestine, large intestine, urethra, or a small incision in the body of the patient. In some situations, the surgeon may approach the target site through both a natural body orifice as well as a small incision in the body.

Remotely controlling distal portions of one or more catheters to precisely position and maintain the position of system components to treat tissue that may lie deep within a patient, e.g., the left atrium of the heart, can be difficult. These difficulties are due, in part, to limited control of movement and articulation of system components and tissues, associated limitations on imaging and diagnosis of target tissue, and limited abilities and difficulties of accurately determining actual positions of system components and distal portions thereof within the patient. For example, it may be difficult to achieve the desired movement resulting from articulation and/or rotation of a particular robotic instrument system component such as a catheter advanced through a sheath and deployed at a desired position. Achieving and maintaining a desired position may also be difficult when external forces applied to a catheter, e.g., when external forces are applied to a distal end of a catheter as a result of contacting tissue. These limitations can complicate or limit the effectiveness of surgical procedures performed using minimally invasive robotic instrument systems.

### SUMMARY

According to one embodiment, a spine apparatus for a flexible, elongate instrument comprises an elongate body having a proximal end and a distal end, and defining a lumen that extends there between. A wall of the elongate body defines a plurality of apertures, and the elongate body comprises a unitary structure that has a plurality of discrete sections, each of which has at least one distinguishing structural attribute that differentiates it from the other sections. A distinguishing attribute of at least one section is related to the plurality of apertures, and a flexibility of the elongate body varies along its length based on the arrangement of the sections.

According to another embodiment, a spine apparatus for a flexible, elongate instrument comprises an elongate body having a proximal end and a distal end and that defines a lumen that extends there between. The elongate body comprises a unitary structure that has a plurality of discrete sections, each of which has at least one distinguishing structural attribute that differentiates it from the other sections. A flexibility of the elongate body varies along its length based on the arrangement of the sections.

According to a further embodiment, a surgical instrument system comprises a flexible, elongate sheath and catheter instruments. The sheath instrument has a sheath body and a plurality of respective control elements that extend through respective lumens defined by the sheath body. The catheter instrument is coaxially positioned within a central lumen defined by the sheath instrument and has a catheter body and a plurality of respective control elements that extend through respective lumens defined by the catheter body. At least one of the sheath and catheter instruments comprises a flexible spine. The spine comprises an elongate body having a proximal end, a distal end and a lumen that extends there between. The elongate body comprises a unitary structure that includes a plurality of discrete sections, each of which has at least one structurally distinguishing attribute such that the flexibilities of the spine and the respective sheath and/or catheter instrument vary along their respective lengths based on the arrangement of the discrete sections.

In accordance with a further embodiment, a flexible, elongate instrument comprises a flexible, elongate sheath body that defines a central lumen configured to receive a catheter of the robotic instrument system and a plurality of control elements extending through respective lumens defined by the sheath body. The sheath body comprises a flexible spine having a proximal end and a distal end and defining a central lumen that extends there between. The spine comprises an elongate body having a unitary structure that includes a plurality of discrete sections, each of which has at least one distinguishing structural attribute that structurally differentiates it from the other sections such that the flexibility of the elongate body varies along its length.

In accordance with another embodiment, a flexible, elongate instrument comprises a flexible, elongate sheath body defining a central lumen configured to receive a catheter of the robotic instrument system and a plurality of control elements extending through respective lumens defined by the sheath body. The sheath body comprises a flexible spine having a proximal end and a distal end and defining a central lumen that extends there between. The spine comprises an elongate body that has a unitary structure that defines a plurality of apertures and includes a plurality of discrete sections. Each section has at least one distinguishing attribute that structurally differentiates it from the other discrete sections. A distinguishing attribute of at least one of the sections is related to the plurality of apertures, and a flexibility of the elongate body varies along its length based on the arrangement of the discrete sections.

According to another embodiment, a flexible, elongate instrument comprises a flexible, elongate catheter body that defines a central lumen configured to receive a working instrument of the robotic instrument system and a plurality of control elements extending through respective lumens defined by the catheter body. The catheter body comprises a flexible spine having a proximal end and a distal end and defining a central lumen there between. The spine comprises an elongate body having a unitary structure that includes a plurality of discrete sections. Each discrete section has at least one distinguishing structural attribute that structurally differentiates it from the other discrete sections such the flexibility of the elongate body varies along its length.

In accordance with yet another embodiment, a flexible, elongate surgical instrument, comprises a catheter body that defines a central lumen configured to receive a working instrument of the robotic instrument system and a plurality of control elements that extend through respective lumens defined by the catheter body. The catheter body comprises a flexible spine having a proximal end and a distal end and defining a central lumen that extends there between. The spine comprises an elongate body having a unitary structure that defines a plurality of apertures and includes a plurality of discrete sections, each of which has at least one distinguishing attribute that structurally differentiates it from the other sections. A distinguishing attribute of at least one of the sections is related to the plurality of apertures, and a flexibility of the elongate body varies along its length based on the arrangement of the discrete sections.

In a further alternative embodiment, a surgical instrument system comprises a flexible, elongate sheath instrument and a catheter instrument. The sheath instrument has a sheath body and a plurality of control elements that extend through respective lumens define by the sheath body. The catheter instrument is coaxially positioned within a central lumen defined by the sheath instrument and has a catheter body and a plurality of control elements that extend through respective lumens defined by the catheter body. At least one of the elongate sheath instrument and the catheter instrument includes a flexible spine, which comprises an elongate body having a proximal end and a distal end and defining a central lumen that extends between there between. The elongate body comprises a unitary structure that defines a plurality of apertures and includes a plurality of discrete sections, each of which has at least one distinguishing structural attribute that structurally differentiates it from the other discrete sections. A distinguishing attribute of at least one of the sections is related to the plurality of apertures, and a flexibility of the elongate body varies along its length based on the arrangement of the discrete sections.

In a further embodiment, a surgical instrument system comprises a flexible, elongate sheath instrument and a catheter instrument. The sheath instrument has a sheath body and a plurality of control elements that extend through respective lumens defined by the sheath body. The catheter instrument is coaxially positioned within a central lumen defined by the sheath instrument and has a catheter body and a plurality of control elements that extend through respective lumens defined by the catheter body. At least one of the elongate sheath instrument and the catheter instrument includes a flexible spine, which has an elongate body that includes proximal end and a distal end and defining a central lumen that extends there between. The elongate body comprises a unitary structure that defines a plurality of I-shaped apertures and a plurality of discrete sections. Each section has at least one distinguishing structural attribute that structurally differentiates it from the other discrete sections. A distinguishing attribute of at least one of the sections is related to the plurality of apertures, and a flexibility of the elongate body varies along its length based on the arrangement of the discrete sections.

In accordance with a further embodiment, a spine apparatus of a flexible, elongate instrument comprises an elongate body having a proximal end and a distal end and defining a lumen that extends there between. The elongate body defining a plurality of apertures and comprises multiple unitary structures. A first unitary structure includes a first plurality of discrete sections, each of which has at least one distinguishing structural attribute that structurally differentiates it from the other sections of the first unitary structure, and a second unitary structure includes a second plurality of discrete sections, each of which has at least one distinguishing structural attribute that structurally differentiates it from the other sections, wherein the flexibility of the elongate body varies along its length.

In one or embodiments having elongate spine bodies that define apertures, a distinguishing structural attribute of at least one discrete section is related to the plurality of apertures and may relate to the existence of apertures, a size, a shape (e.g., length or subtended angle, width), a number, a spacing, and/or a degree of overlap of apertures defined by a section. For example, apertures in different sections may have different sizes, and a first section of an elongate body may define apertures that are longer and wider than apertures defined by a second discrete section proximal to the first discrete section, and apertures defined by the second discrete section may be longer and wider than apertures defined by a third discrete section proximal to the second discrete section. As another example, a distinguishing structural attribute related to the plurality of apertures comprises a shape of a middle portion of the respective apertures, which may be I-shaped apertures, which may include an enlarged or bulbous middle portion, respective intermediate portions extending from respective ends of the middle portion, and respective end portions extending from the respective intermediate portions. In one embodiment, the intermediate portions are the narrowest portions of the I-shaped apertures, whereas the respective middle and end portions have approximately same widths.

In one or more embodiments apertures may have a symmetrical shape and be arranged in a symmetrical formation. Different sections may also have different aperture attributes, e.g., different sizes, numbers, overlap, spacing, etc.

In one or more embodiments, structural differences between discrete sections are defined relative to the sections having the same length.

In one or more embodiments, a structural attribute that distinguishes discrete sections comprises a material or other material attribute such as a density of a material, e.g., a density of a braid material that is used in an elongate spine body, and a dimension (e.g., wall thickness, width, tapering width, length). Certain sections may be made or formed from the same material but be structurally distinguished on other bases, e.g., size, shape, etc.

In one or more embodiments, a spine apparatus may be comprised of multiple spine structures. In one embodiment, a sheath and/or catheter instrument includes two unitary structures, each of which has proximal end and a distal end and defining a central lumen that extends there between. The unitary structure includes a plurality of discrete sections, each of which has at least one distinguishing attribute that structurally differentiates it from the other respective sections of the respective spine. In certain embodiments, the spines may be made or formed of the same material but be different sizes. Multiple spines may partially overlap, completely overlap as stack of spine structures, and/or be arranged end-to-end in a non-overlapping manner.

### BRIEF DESCRIPTION OF THE DRAWINGS

The forgoing and other aspects of embodiments will be understood with reference to the following detailed description, in conjunction with accompanying drawings, which illustrate the design and utility of various embodiments, and in which like reference numbers identify corresponding components throughout, wherein:

FIG. 1 illustrates an embodiment of a flexible instrument assembly that includes controllable and torquable sheath and catheter instrument having spines constructed according to embodiments;

FIG. 2 illustrates a splayer assembly and how controllable and torquable sheath and catheter instruments shown in FIG. 1 are coaxially arranged through respective splayers;

FIG. 3 illustrates one manner in which instruments shown in FIGS. 1-2 may be utilized for diagnosis or treatment of endocardial tissue;

FIG. 4A is a cross-sectional view of a portion of a sheath instrument having a spine constructed according to one embodiment;

FIG. 4B is a cross-sectional view of a portion of another embodiment of a sheath assembly having a spine, braided layers and a central lumen having a different key shape compared to the embodiment shown in FIG. 4A;

FIGS. 4C-G illustrates alternative lumen or key configurations that may be utilized with embodiments;

FIG. 5A is a perspective view of an embodiment of a flexible and torquable spine apparatus for use in a sheath instrument and having an elongate body that has a unitary structure including a plurality of discrete sections and variable flexibility;

FIG. 5B illustrates the elongate body of the spine apparatus illustrated in FIG. 5A in an unrolled or pre-formed state;

FIG. 5C illustrates a spine apparatus of a sheath instrument and associated control ring and soft distal tip components;

FIGS. 6A-D illustrate I-shaped apertures defined by different discrete sections of one embodiment of a spine apparatus shown in FIGS. 5A-B, wherein the I-shaped apertures have an enlarged or bulbous middle portion and narrow or tapered intermediate portions;

FIGS. 7A-B illustrate an I-shaped aperture of a different shape of another embodiment of a spine apparatus, wherein the I-shaped aperture has a narrow middle portion;

FIG. 8A is a cross-sectional view of a portion of a catheter instrument having a spine constructed according to one embodiment;

FIG. 8B is cross-sectional view of a portion of another embodiment of a catheter instrument having a spine, braided layers and an outer surface having a different key shape;

FIG. 9A is a perspective view of an embodiment a flexible and torquable spine apparatus for use in a catheter instrument having an elongate body that has a unitary structure including a plurality of discrete sections and variable flexibility;

FIG. 9B illustrates the elongate body of a spine apparatus illustrated in FIG. 9A in an unrolled or pre-formed state;

FIG. 9C illustrates a spine apparatus of a catheter instrument and associated control ring and soft distal tip components;

FIG. 10 is a cross-sectional view of a portion of another embodiment of a catheter instrument having a spine apparatus;

FIGS. 11A-B are cross-sectional views of a further embodiment of a portion of a catheter instrument having a spine apparatus;

FIG. 12 generally illustrates a distal portion of a sheath instrument and/or a catheter instrument and how the flexibility of discrete sections of a spine may vary such that the flexibility along the length of an instrument varies, wherein a distal portion of the instrument is more flexible than a proximal portion of the instrument;

FIG. 13 generally illustrates a distal portion of a sheath instrument and/or a catheter instrument and how the flexibility of discrete sections of a spine may vary such that the flexibility along the length of an instrument varies, wherein an intermediate portion of the instrument is more flexible than a proximal portion of the instrument;

FIG. 14 illustrates one embodiment of a flexible and torquable support or spine apparatus for use in a sheath and/or catheter instrument and that includes at least one discrete section having a different dimension than a dimension of other sections;

FIG. 15 illustrates one embodiment of a flexible and torquable support or spine apparatus for use in a sheath and/or catheter instrument and that includes at least one discrete section having a different length than a length of other sections;

FIG. 16 illustrates one embodiment of a flexible and torquable support or spine apparatus for use in a sheath and/or catheter instrument and that includes at least one discrete section having a different wall thickness than a wall thickness of other sections;

FIG. 17A illustrates one embodiment of a flexible and torquable support or spine apparatus for use in a sheath and/or catheter instrument and that includes at least one discrete section having a different width or diameter than the width or diameter of other sections;

FIG. 17B is a cross-sectional view of one embodiment of a flexible and torquable support or spine apparatus for use in a sheath and/or catheter instrument and that includes a distal discrete section having a different width or diameter than a more proximal section;

FIG. 17C is a partial side view of one embodiment of a flexible and torquable support or spine apparatus for use in a sheath and/or catheter instrument and that includes at least one discrete section having a different width or diameter than other sections;

FIG. 18 is a cross-sectional view of one embodiment of a flexible and torquable support or spine apparatus for use in a sheath and/or catheter instrument and that includes discrete sections of the same length, at least one discrete section being structurally distinguished from the other sections based on at least one other structural attribute;

FIG. 19 illustrates one embodiment of a flexible and torquable support or spine apparatus for use in a sheath and/or catheter instrument and that includes at least one discrete section that is made of a different material or has a different material attribute than the material or material attribute of other sections;

FIG. 20 illustrates one embodiment of a flexible and torquable support or spine apparatus for use in a sheath and/or catheter instrument and that includes at least one discrete section having a different number of apertures than the number of apertures of other sections;

FIG. 21 illustrates one embodiment of a flexible and torquable support or spine apparatus for use in a sheath and/or catheter instrument and that includes at least one discrete section that includes apertures of a different size than apertures of other sections;

FIG. 22 is a graph illustrating how aperture size may vary along the length of an embodiment of a flexible and torquable support or spine apparatus and along a corresponding portion of a sheath and/or catheter instrument;

FIG. 23 illustrates one embodiment of a flexible and torquable support or spine apparatus for use in a sheath and/or catheter instrument and that includes at least one discrete section that includes apertures of a different spacing compared to aperture spacing of other sections;

FIG. 24 illustrates one embodiment of a flexible and torquable support or spine apparatus for use in a sheath and/or catheter instrument and that includes at least one discrete section that includes apertures that overlap by a different amount or degree than aperture overlap of other sections;

FIG. 25 illustrates one embodiment of a flexible and torquable support or spine apparatus for use in sheath and/or catheter instrument wherein a distinguishing structural attribute is an existence of apertures within a discrete section;

FIG. 26 illustrates one embodiment of a flexible and torquable support or spine apparatus for use in a sheath and/or catheter instrument and that includes discrete sections having different numbers of distinguishing attributes;

FIG. 27 illustrates one embodiment of a flexible and torquable support or spine apparatus for use in a sheath and/or catheter instrument and that includes at least one discrete section that has a different diameter than other sections;

FIG. 28 illustrates one embodiment of a flexible and torquable support or spine apparatus for use in a sheath and/or catheter instrument that includes at least one discrete section that is structurally distinguished from other sections based on diameter and aperture sizes;

FIG. 29 illustrates one embodiment of a flexible and torquable support or spine apparatus for use in a sheath and/or catheter instrument that includes at least one discrete section that is structurally differentiated from other sections based on aperture shape and section material;

FIG. 30 illustrates one embodiment of a flexible and torquable support or spine apparatus for use in a sheath and/or catheter instrument that includes at least one discrete section that is structurally differentiated from other sections based on aperture size and overlap;

FIGS. 31A-N and FIGS. 32A-G illustrate a robotic surgical system and components and applications thereof that may include or be utilized with spine embodiments, wherein FIG. 31A illustrates a robotic medical instrument system, FIG. 31B illustrates a setup joint or support assembly, FIG. 31C illustrates an operator workstation including a master input device and data gloves, FIG. 31D is a block diagram of a system architecture of a robotic medical instrument system in which embodiments may be implemented or with which embodiments may be utilized, FIG. 31E illustrates a sheath instrument and associated sheath splayer, FIG. 31F illustrates a catheter instrument and associated catheter splayer, FIG. 31G illustrates the catheter instrument shown in FIG. 31F coaxially positioned within the sheath instrument shown in FIG. 31E, FIG. 31H is a perspective view of an instrument driver for use with the splayers and instrument assemblies shown in FIGS. 31E-G, FIG. 31I illustrates examples of motors in splayers that may be controlled or actuated by an instrument driver to controllably articulate or manipulate associated sheath and catheter instruments, FIGS. 35J-N illustrate different ways in which sheath and catheter instruments can be manipulated, FIGS. 32A-E illustrate how distal portions of sheath and guide instruments may be navigated through vasculature of a patient to a target site such as a site within the patient's heart, FIG. 32F generally illustrates a distal portion of a catheter constructed according to one embodiment that forms an arc with a substantially constant radius of curvature, and FIG. 32G illustrates a distal portion of a catheter instrument constructed according to one embodiment that is bendable into an J-shape having a small radius of curvature.

### DETAILED DESCRIPTION OF THE ILLUSTRATED EMBODIMENTS

Embodiments of the invention are related to a flexible spine for use in one or more surgical instruments including a catheter and/or sheath of a robotic instrument system. The spine includes an elongate body that defines a central lumen and is a unitary structure that includes a plurality of discrete sections, each of which has at least one distinguishing structural attribute that differentiates it from other sections. Discrete sections may be assembled as, or are formed as, a unitary structure. Such distinguishing structural attributes may include, for example, materials, material attributes, shapes, sizes and/or attributes related to apertures in a wall of the elongate body, such as a number, shape, size, spacing and degree of overlap of such apertures, and combinations thereof. The arrangement of discrete, structurally different sections results in varying flexibility of the elongate spine and of corresponding sections of a surgical instrument incorporating the spine.

Instruments that include such flexible and torquable spine structures have variable flexibility along their lengths and can transmit torque to accurately position and maintain the position of instruments in the presence of external forces. For example, certain embodiments allow for accurate positioning of sheath and/or catheter instruments according to the controlled articulation, deflection or manipulation of such instruments as a result of variable flexibility provided by the support apparatus or spine, while also allowing torque or twisting forces to be properly transmitted through the spine. In this manner, a distal portion of an instrument can be manipulated and positioned in its intended location and may be maintained at a particular location in that position in the presence of external forces that may be applied to the distal portion, e.g., by tissue, such as when a distal end of the instrument engages tissue, or when tissue moves to engage or press against the distal end of the instrument.

A discrete section of a spine is defined based on a distinguishing structural attribute that structurally differentiates that discrete section from other discrete sections such that the flexibility along the unitary structure varies along its length and also provides for transmission of torque or twisting forces. In one embodiment, a discrete section of a spine and a corresponding section of an elongate instrument may be more flexible than a more proximal discrete section of the spine and corresponding section of the elongate instrument.

A discrete section that is defined based on a combination of structural attributes may be defined based on combinations of two, three, four and other numbers of structural attributes. Further, certain embodiments may involve use of one or multiple spines or spine elements, which may be arranged end to end or partially or completely overlap each other, e.g., sections of two, three or other numbers of elongate spine bodies may overlap. In this manner, one or multiple spine structures may be utilized to alter the manner in which flexibility varies and how torque or twisting forces are transmitted along the structure. Further, embodiments utilize discrete sections that define symmetrical apertures and/or symmetrical formations such that articulation and torque transmission can be achieved in multiple directions in a consistent or symmetrical t manner.

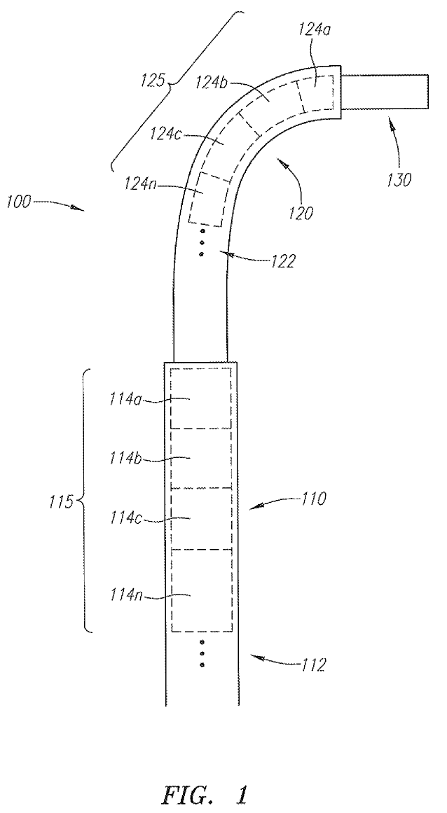

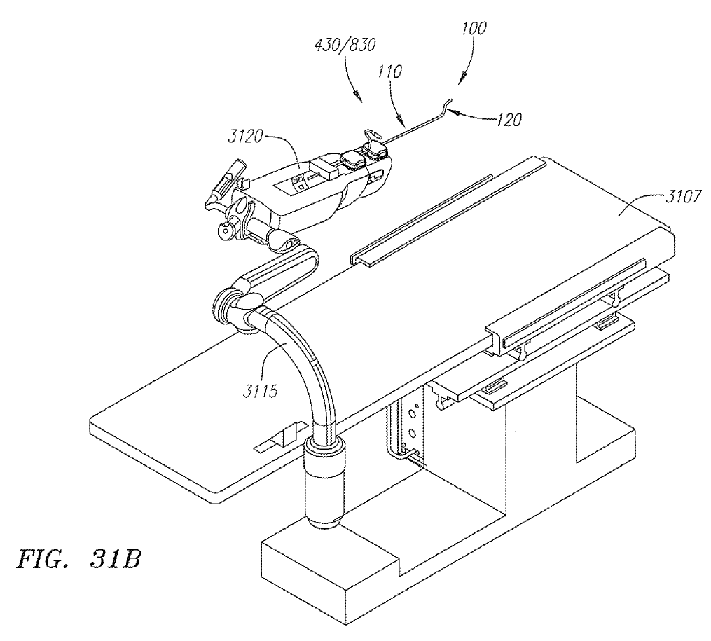

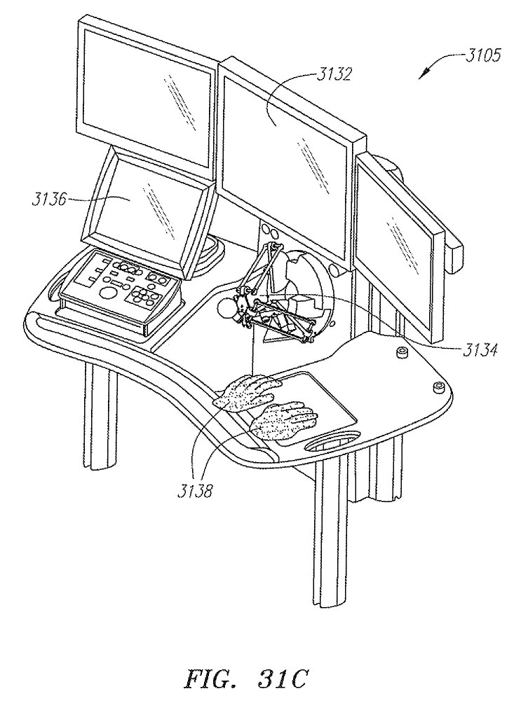

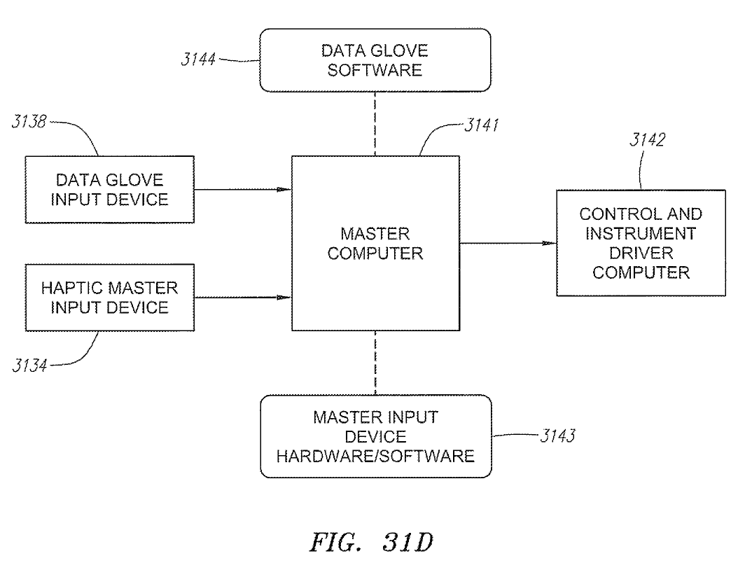

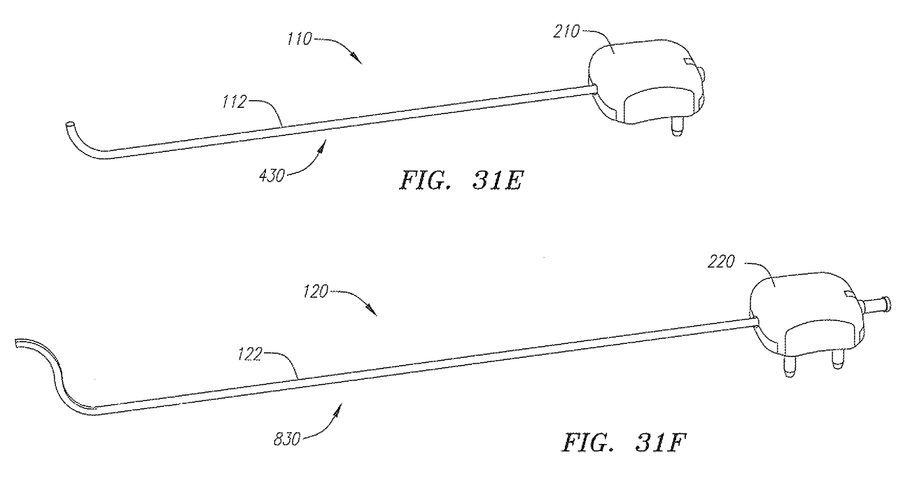

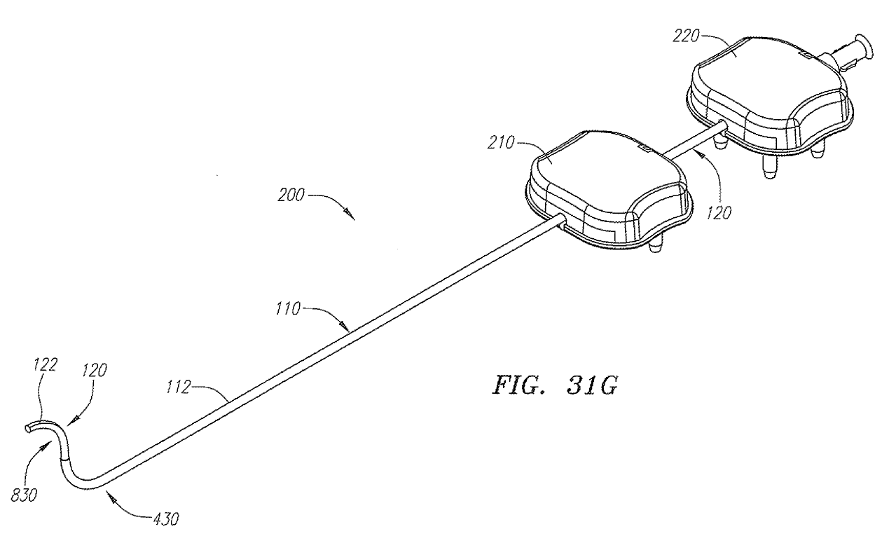

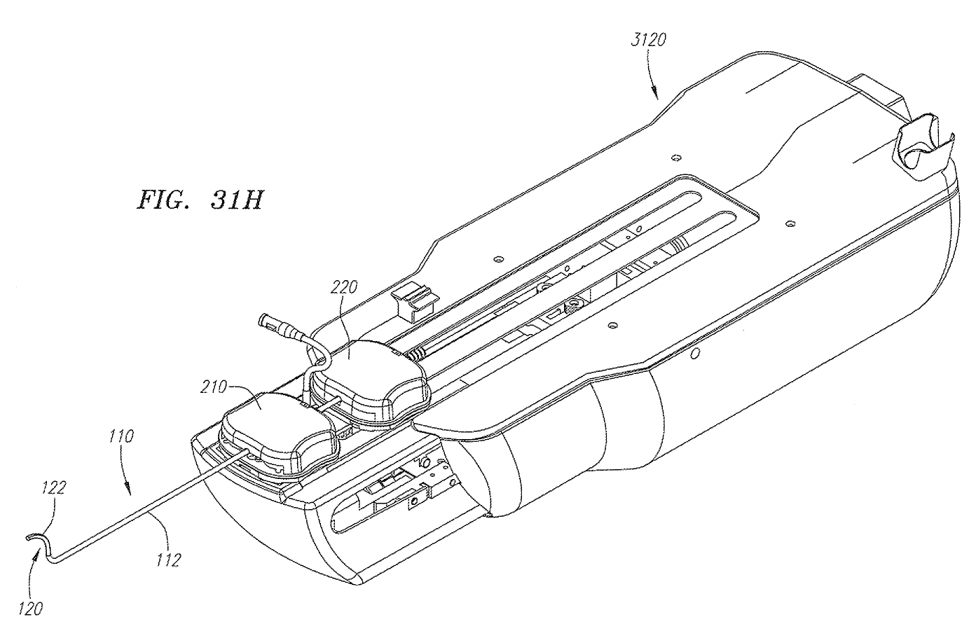

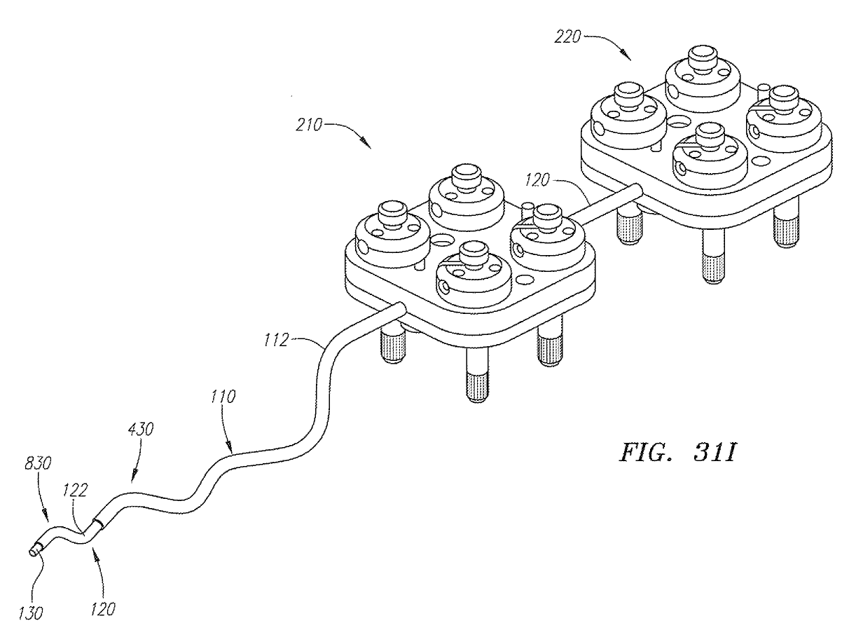

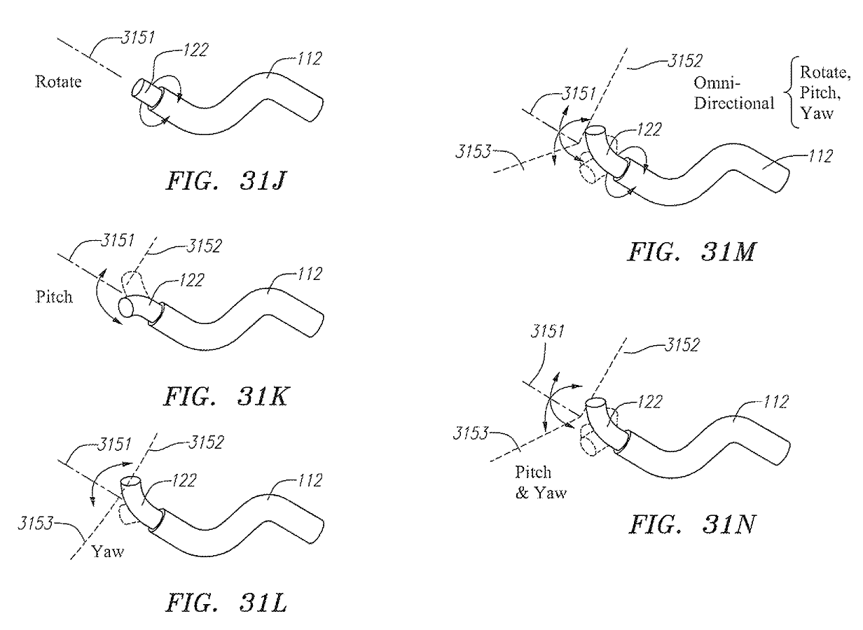

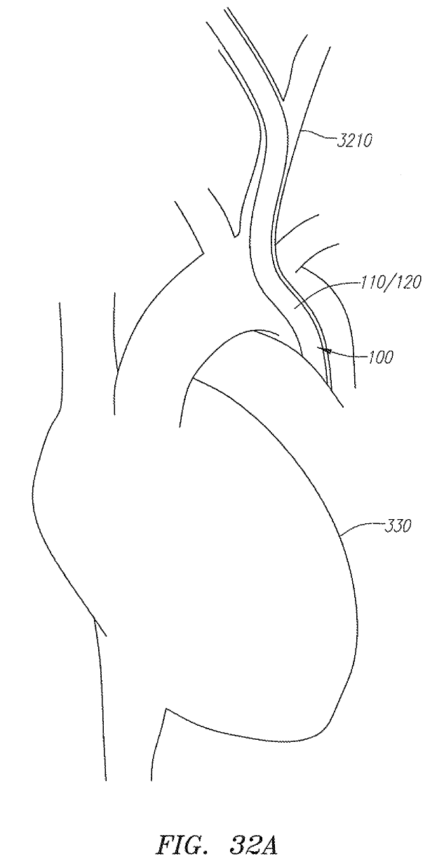

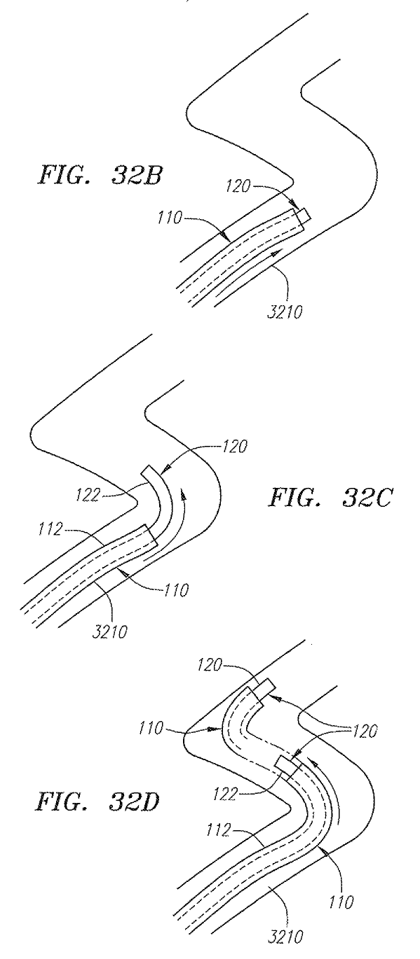

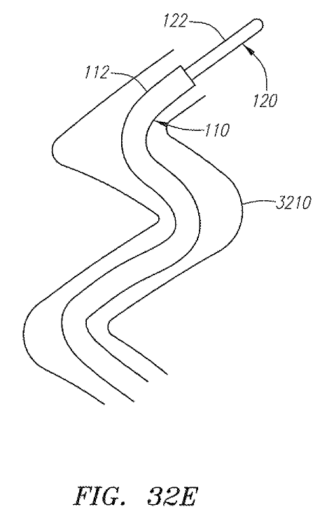

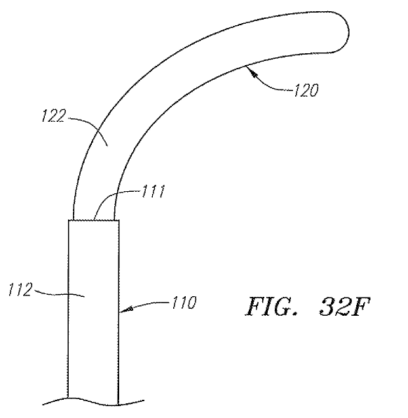

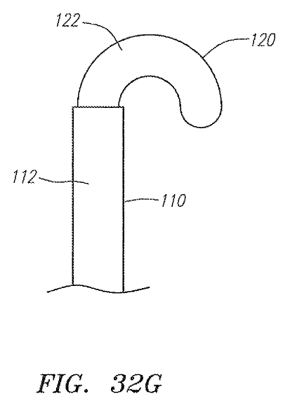

Aspects of spine embodiments and components and applications thereof are described with reference to FIGS. 1-30. Examples of robotic surgical systems and components thereof that may include or be utilized with embodiments and their applications are described in further detail with reference to FIGS. 31A-32G.

Referring to FIG. 1, one embodiment of an instrument assembly 100 for use in a robotic surgical system includes an elongate sheath instrument 110 (referred to as sheath or sheath instrument 110) and an elongate catheter instrument 120, such as a guide catheter (referred to as a catheter or catheter instrument 120). During use, a working instrument 130 is advanced through or coupled to a distal end of the catheter instrument 120 to a target site.

As shown in FIG. 1, an elongate portion 112 of the sheath 110, which may be a distal portion, includes a spine 115 in the form of an elongate body having a unitary structure comprised of a plurality of discrete sections 114a-n (generally referred to as discrete section 114). An elongate portion 122 of the catheter 120, which may also be a distal portion, is configured for advancement through the sheath instrument 110 and may also include a spine 125 in the form of an elongate body that is a unitary structure comprised of a plurality of discrete sections 124a-n (generally referred to as discrete section 124). Spine structures 115, 125 (illustrated in phantom) for use in sheath and catheter instruments 110, 120 may be formed by assembly of multiple discrete sections 114, 124, or formed as a result of manipulation of a unitary element to form discrete sections 114, 124 depending on the manufacturing method employed.

In one embodiment, in which each of the sheath and catheter instrument 110, 120 includes respective spines 115, 125, at least one discrete section 114, 124 has at least one distinguishing attribute that structurally differentiates it from the other sections 114, 124. In this manner, the spine 115, 125 formed by the collection of discrete sections 114, 124 result in unitary spine structures 115, 125 having variable flexibility along their respective lengths while also providing torque transmission.

Although FIG. 1 illustrates both of the sheath and catheter instruments 110, 120 having respective unitary spine structures 115, 125, in another embodiment, only the sheath instrument 110 includes a spine 115 that includes a plurality of discrete sections 114a-n to provide variable flexibility, and in another embodiment, only the catheter instrument 120 includes a spine 125 that includes plurality of discrete sections 124a-n to provide variable flexibility. Further, although FIG. 1 generally illustrates unitary spine structures 115, 125 including three discrete sections 114a-c, 124a-c, a spine may actually be integrated within a sheath or catheter 110, 120, as will be described in further detail below. Further, a spine may include or be formed to include other numbers (n) and arrangements of discrete sections 114, 124 and may extend along different lengths of a sheath or catheter instrument, e.g., only along a distal portion (as generally illustrated in FIG. 1) or along longer lengths of an instrument as necessary. Thus, FIG. 1 is provided to generally illustrate one manner in which embodiments may be implemented in a non-limiting manner, and it should be understood that spines structures 115, 125 may include two, three, four, ten, twenty, and other numbers of discrete sections 114, 124 and may extend various lengths and be integrated within or a part of a sheath and/or catheter instrument 110, 120.

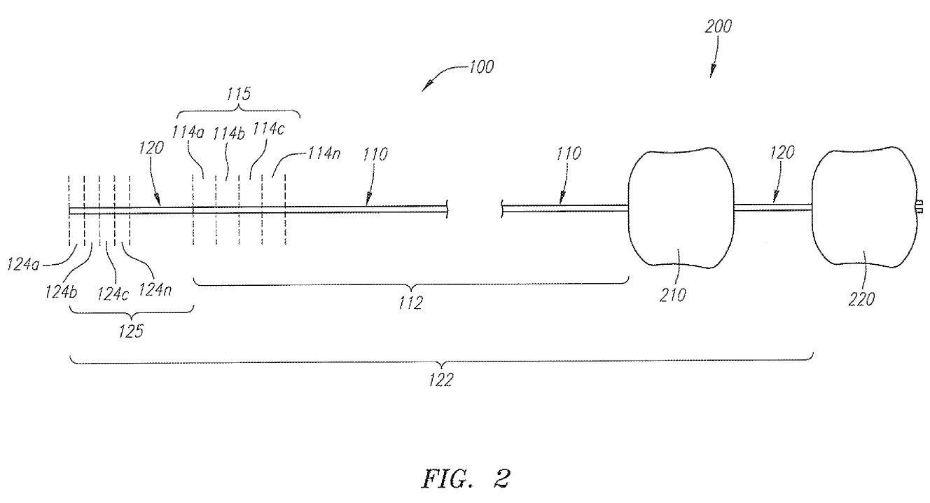

Referring to FIG. 2, an instrument assembly 200 configured for use in a robotic instrument system includes sheath and catheter instruments 110, 120 that are operably coupled together in a coaxial manner by respective splayers or drive elements 210 and 220 (generally splayers 210, 220). Each splayer 210, 220 includes associated motors or drivers (not illustrated in FIG. 2) that drive and controllably articulate elongate portions 112, 122 of the respective sheath and catheter instruments 110, 120. One example of such a system is a robotic surgical system known as the Sensei™ Robotic Catheter System, which is available from Hansen Medical, Inc., Mountain View, Calif. Splayers 210, 220 may also be used for controlling or driving other devices, e.g., such as a laser, a basket and other components, and may be used in navigation applications.

The elongate portion 122 of the catheter instrument 120 is advanced or fitted through a central lumen defined by the elongate portion 112 of the sheath instrument 110. The elongate portions 112 and/or 122 may be steered or navigated substantially as a unit by the splayers 210, 220 of the respective sheath and catheter instruments 110, 120. For this purpose, an instrument driver (not illustrated in FIG. 2) operates motors or mechanisms in the splayers 210, 220 to controllably bend, articulate, or steer, and navigate the respective elongate portions 112, 122 of the sheath and the catheter instruments 110, 120. The elongate portion 112 of the sheath 110 and the elongate portion 122 of the catheter 120 may also be steered or navigated separately by the respective splayer units 210, 220 as operated by the instrument driver.

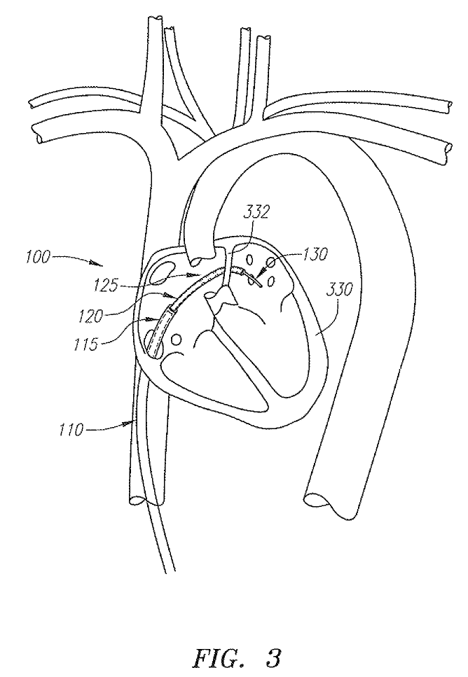

Referring to FIG. 3, one application of the assemblies and components shown in FIGS. 1-2 is to diagnose or treat endocardial tissue. FIG. 3 depicts delivery of the instrument assembly 100 utilizing a standard atrial approach in which the robotically controlled sheath and catheter instruments 110, 120 having respective spines 115, 125 (illustrated in phantom) are advanced through the inferior vena cava and into the right atrium of the heart 330. An image capture device (not illustrated in FIG. 1), such as an endoscope or intra-cardiac echo (“ICE”) sonography catheter, may be advanced into the right atrium to provide a field of view of the interatrial septum 332. The catheter 120 may be driven to the septum wall 332, and the septum 332 may be crossed using a conventional technique of first puncturing the fossa ovalis location with a sharpened device, such as a needle or wire, which is passed through a working lumen of the catheter 120. A dilator or other working instrument 130 can then be passed over the sharpened device, thereby leaving dilator 130, over which the catheter 120 may be advanced. Various other working instruments 130 may be delivered through or attached to the catheter 120 as necessary and depending on the surgical application. For example, for treatment of atrial fibrillation, the working instrument 130 may be an ablation catheter that delivers targeted radio frequency (RF) energy to selected endocardial tissue.

Certain embodiments allow for accurate positioning of sheath and/or catheter instruments 110, 120 that may be used for these and other purposes according to the controlled articulation, deflection or manipulation of the sheath and/or catheter instruments 110, 120 through tortuous vasculature. These abilities are achieved by variable flexibility of an instrument as provided by a support or spine structure 115, 125 that allows the instrument to bend or be articulated, while also providing for transmission of torque or twisting forces. Further aspects of such components and other aspects of the robotic surgical system and applications thereof are described with reference to FIGS. 31A-32G, and in various applications previously incorporated herein by reference.

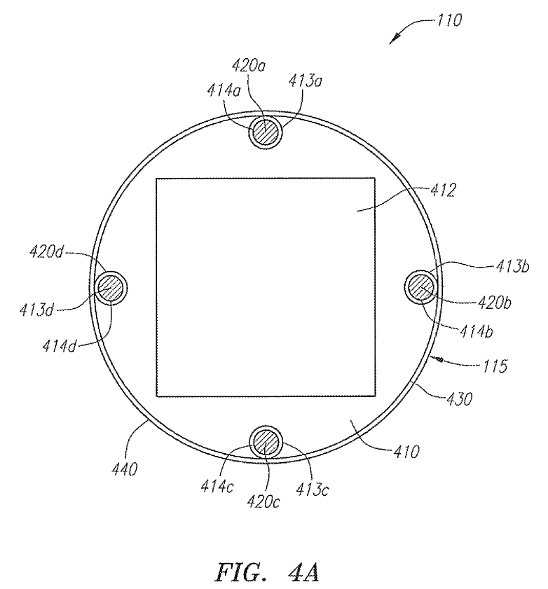

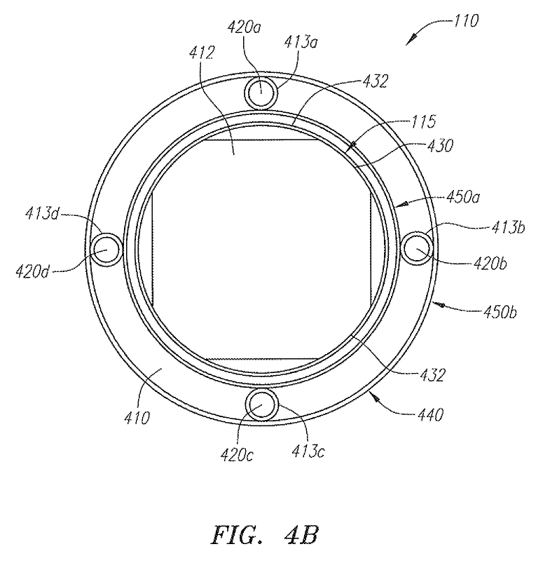

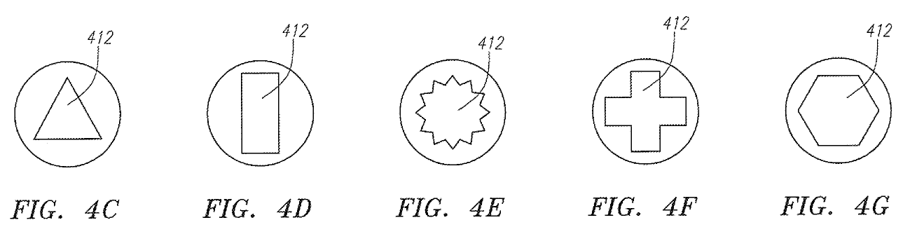

Referring to FIGS. 4A-B, a sheath instrument 110 constructed according to certain embodiments includes an elongate portion 112 that may have a length of about 68 cm, or about 27″, includes a sheath body 410, which may be generally tubular in shape and made of a polymeric material such as PTFE, PTFE doped polyimide, or another suitable polymer. The sheath body 410 may be formed by a molding process or other suitable process. The polymeric material may be substantially flexible, such that it may be bent upwardly and downwardly, steered (pitch or yaw), and/or rotated relatively freely without substantial resistance or constraint.

The sheath body 410 defines a central lumen 412 that is configured to receive an elongate portion 122 of the catheter instrument 120, which may, for example, have a length of about 88 cm, or about 35″. In the embodiment illustrated in FIG. 4A, the central lumen 412 of the sheath body 410 is a “keyed” lumen having square shape configured to interface with a catheter instrument 120 having a corresponding shape such that the elongate portion 122 of the catheter 120 does not rotate or does so to a minimal or small degree within the keyed central lumen 412 of the sheath 120. Other key shapes may be utilized, including a square with rounded corners (FIG. 4B) triangle (FIG. 4C), a rectangle (FIG. 4D), a star (FIG. 4E), an “X” or “cross” shape (FIG. 4F) a polygon (FIG. 4G), e.g., a hexagon, and other shapes. Thus, various “keyed” configurations may be utilized in embodiments.

In one system, the elongate portion 122 of the catheter 120 has an outer diameter of about 4 French to about 7 French, and the central lumen 412 of the sheath body 410 has an inner diameter or diagonal measurement of about 0.053″ (4 French) to about 0.092″ (7 French) to accommodate the elongate portion 122. The inner surface of the central lumen 412 may be substantially smooth such that a mating body such as the elongate portion 122 of the catheter 120 may slide or move along the lumen 412 without significant frictional resistance.

The sheath body 410 also includes a plurality of tubes 413a-d (generally 413) that define a plurality of smaller lumens 414a-d (generally 414) that are configured to receive or accommodate respective control elements 420a-d such as stainless steel wires (generally 420). The sheath 110 may be structured such that the center-to-center distance between opposing control element lumens 414a and 414c, and 414b and 414b, is about 0.11″. The control wires 420 are manipulated to steer the distal section of the elongate portion 112 of the sheath 110 in pitch, yaw, and rotational movements, e.g., to navigate the elongate portion 112 of the sheath 110 through tortuous natural body pathways (arteries, veins, etc.) in minimally invasive surgical procedures. For this purpose, a control wire 420 may have an outer diameter of about 0.0075″, and an inner diameter of a tube 413 that defines a control wire lumen 420 may have an inner diameter or width of about 0.010″ to about 0.012″ to accommodate a control wire 420. Other wire and lumen sizes, including smaller wire and lumen sizes, may also be utilized depending on, for example, the configuration and sizes of other components.

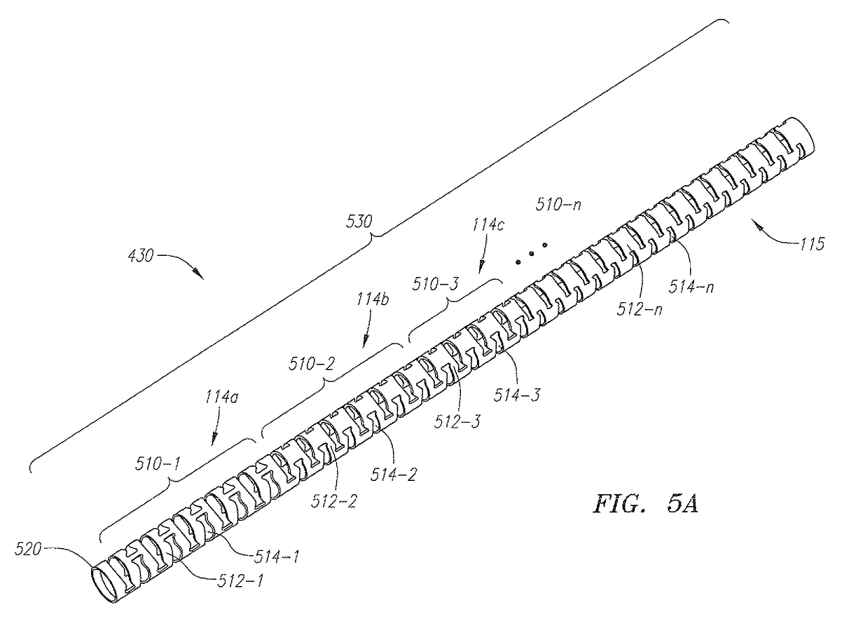

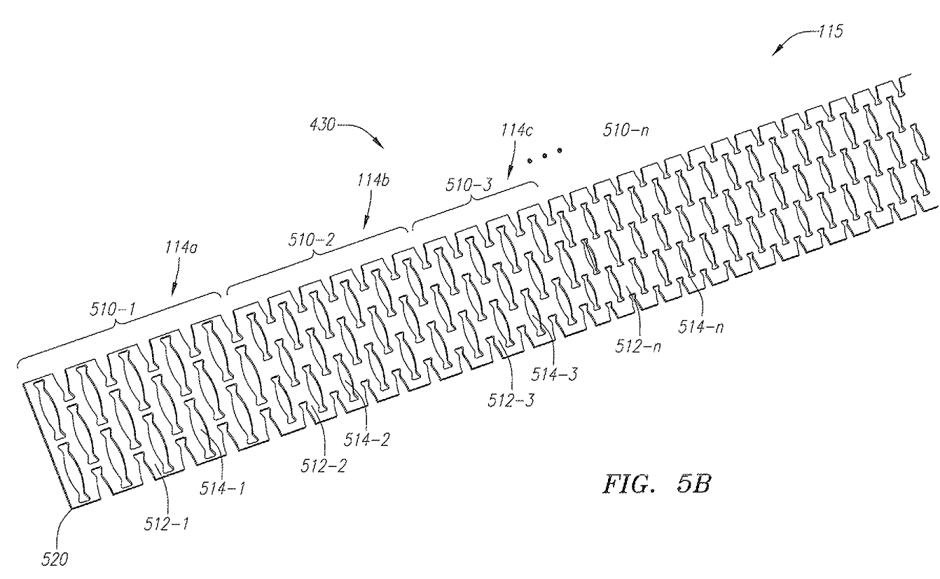

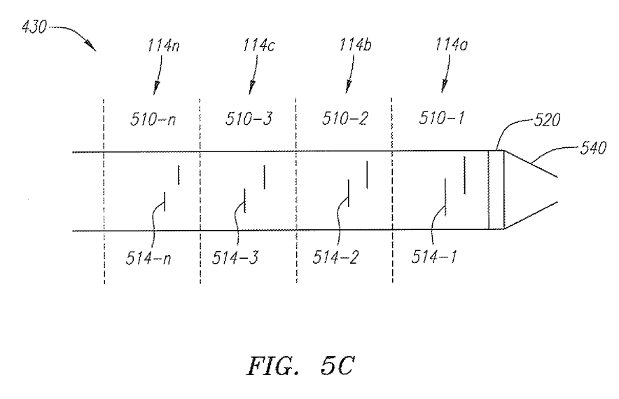

The elongate portion 112 of the sheath 110 also includes a flexible and torquable apparatus or spine 430 (generally referred to as spine 430), which surrounds or encloses the polymer material 410, tubes 413 that define control element lumens 414, and the central lumen 412. The spine 430 may extend along a portion (e.g., along a distal portion) of an outer portion of the elongate portion 112 of the sheath 110 or along the entire length of the elongate portion 112 of the sheath 110. The wall thickness of the spine 430 may be about 0.003″, an inner diameter of the spine 430 may be about 0.090″, and an outer diameter of the spine 430 may be about 0.096″. An inner lining 432 may also be applied to an inner surface of the spine 430 to enclose or coat the spine 430. The lining 432 may have a thickness of about 0.0005″ to about 0.002″ and have an inner diameter of about 0.088″. Control wires 420 may be attached to a control ring (not shown in FIG. 4, but one example of which is illustrated in FIGS. 5B-C) that is coupled to a distal portion of the spine 430. Alternatively, control wires 420 may be attached directly to distal portions of the spine 430.

In the illustrated embodiment, the elongate portion 112 of the sheath 110 also includes an outer jacket or cover 440 that encases all of the aforementioned structures into a sheath unit or assembly 110. For this purpose, the cover 440 may be constructed, fabricated, or formed from a flexible and bio-compatible material for use in the human body. For example, the cover 440 may be formed from a polymeric material such as urethane, poly-urethane, nylon, Prebax™, etc. The cover 440 may have a thickness of about 0.026″ an inner diameter or diagonal measurement of about 0.092″ (7 fr)″, and an outer diameter or diagonal measurement of about 0.118 inches (9 French) to about 0.131 inches (10 French), e.g. about 0.126″.

As illustrated in FIG. 4B, in another embodiment, the spine 430 may be positioned inwardly relative to the tubes 413 and control elements 420. FIG. 4B also illustrates that braided layers 450a, 450b (generally 450) may be provided along the inner and outer surfaces of a tube 413 that defines a control wire lumen 420 for the purpose of altering bending, torque, axial (column strength), and radial (kink resistance) strength properties. The outer braided layer 450b may be a part of the jacket or cover 440 and may provide support for control wires and lumens during articulation to prevent detachment or delamination of the control wires 420 and/or tubes 413. The braided layers 450 may be made of a stainless steel or a polymeric material such as Vectran®, nylon or Kevlar®.

Referring to FIGS. 5A-C, one embodiment of a spine 430 for use in a sheath instrument 110 includes a plurality of discrete or structurally distinct sections 510-1 to 510-n (generally referred to as discrete section 510). A control ring 520 is attached to, or a part of, a distal most discrete section 510-1. As discussed with reference to FIGS. 4A-B, control wires 420 may be attached to the control ring 520. The discrete sections 510 collectively form a unitary structure 530 that has variable flexibility along its length. Embodiments provide a sheath instrument 110 that is flexible and torquable through the use of discrete sections 510 that have different structural attributes such that the elongate portion 112 of the sheath can be controllably manipulated and positioned even in the presence of twisting forces that may be generated as a result of articulation of the elongate portion 112.

In the illustrated embodiment, each discrete section 510 includes segments (512-1, 512-2, 512-3, . . . 512-n) (generally 512), between which are defined apertures, gaps or spaces (514-1, 514-2, 514-3, . . . , and 514-n) (generally 514). Apertures 514 may be cut from material that forms a discrete section 512, or apertures 514 may be formed by cutting apertures 514 from unrolled or flat material that is later formed or rolled into the configuration as shown in FIG. 5 or utilizing other suitable fabrication methods. The spine 430 may include a soft distal tip 540 (as shown in FIG. 5C) that may be constructed from a soft polymeric material to facilitate advancement through tortuous vasculature. In the illustrated embodiment, the soft distal tip 540 is attached to the control ring 520 or the distal tip of the spine 430 and the cover 440. Although FIGS. 5A-C illustrate three distinct discrete sections 510, other embodiments may include other numbers (n) of discrete sections, e.g., two, four, ten, twenty, and other numbers (n) of discrete sections 510. Thus, FIGS. 5A-C are provided as one example of how embodiments may be implemented.

In the illustrated embodiment, the spine 430 comprises an elongate body that includes, or is formed to have, a unitary structure 530 having discrete sections 510. In the illustrated embodiment, the unitary structure has a tube-like shape. According to one embodiment, each section 510 has at least one distinguishing structural attribute that differentiates it from the other sections 510. In this manner, a structural attribute defines a dividing line, or the beginning or end of a discrete section 510. With this configuration, the spine 430 (and a corresponding section of an instrument having the spine 430) has variable flexibility along its length based on the arrangement of the discrete sections 510. The arrangement and configuration of the discrete sections 510 that form the unitary structure 530 result in a torquable unitary structure 530 having variable flexibility that allows the elongate portion 112 of the sheath 110 to be positioned in its intended position as determined by manipulation of one or more control wires 420.

According to one embodiment, the flexibility of the spine 430 and the flexibility of the corresponding sections of the elongate portion 112 of the sheath 110 increase from a proximal portion to a distal portion such that the distal end of the spine 430 (discrete section 510-1 in the illustrated embodiment), is the most flexible discrete section 510. In other embodiments, an intermediate discrete section, e.g., one or more of discrete sections 510-2 and 510-3, may be less flexible than another discrete section 510 such that the flexibility varies along the length of the spine 430, but does not increase from the proximal end to the distal end. For ease of explanation, reference is made generally to a spine 430 having flexibility that increases from its proximal end to its distal end.

Certain figures illustrate embodiments of an elongate portion 112 of a sheath 110 that includes a single spine 430, but in other embodiments, an elongate portion 112 of a sheath 110 may include multiple spines 430a-n (generally 430n). According to one embodiment, an elongate portion 112 of a sheath 110 includes multiple spines 430n that partially or completely overlap each other. According to another embodiment, a sheath 110 may include an elongate portion 112 having spines 430n that are arranged end-to-end. Further, in another embodiment, an elongate portion 112 of a sheath 110 may include two or more spines 430n that partially or completely overlap, and other spines 430n that are arranged end-to-end.

Accordingly, Figures that illustrate an elongate portion 112 of a sheath 110 that includes a single spine 430 are provided for ease of explanation and illustration, and it should be understood that other embodiments may involve a sheath 110 having multiple spines 430n, which may or may not be the same size, and which may or may not be overlapping. Further, each spine 430 can have respective pluralities of discrete sections 510, each of which has at least one distinguishing structural attribute that differentiates it from the other discrete sections 510 of the respective spine to provide variable flexibility while also providing desired torque transmission.

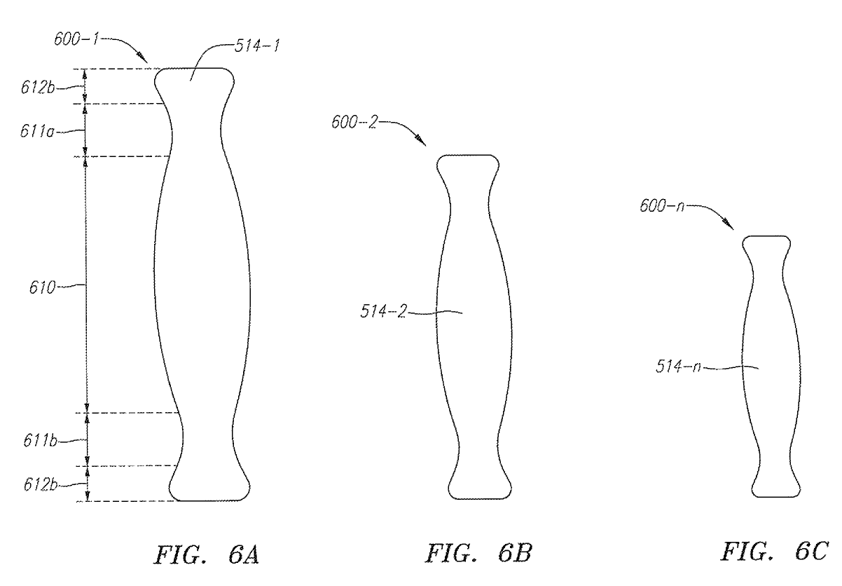

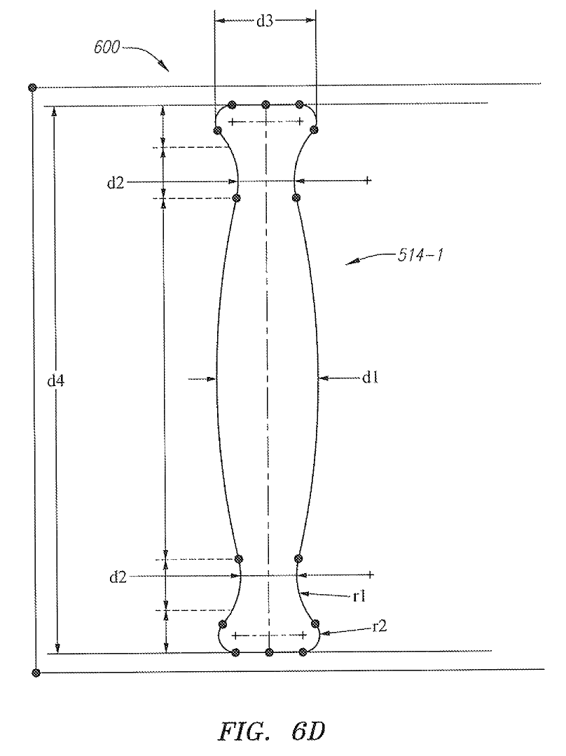

With further reference to FIGS. 6A-D, according to one embodiment, the elongate body of a spine 430 may define apertures 514a-n. In the illustrated embodiment, the apertures 600-1 to 600-n (generally 600) have a symmetrical shape and are arranged in a symmetrical formation, as shown in FIGS. 5A-B. Embodiments utilize symmetrical aperture 600 configurations and formations to provide for symmetrical flexibility and symmetrical torque transmission in both rotational directions, in contrast to certain asymmetrical apertures, such as L-shaped apertures.

In the illustrated embodiment, an aperture 600 has an expanded or enlarged I-shape, or a “double-ended vase” shape, and has an enlarged or bulbous middle portion 610, intermediate portions 611a and 611b (generally 611) extending from and adjacent to the respective ends of the middle portion 610, and end portions 612a and 612b (generally 612) extending from and adjacent to the respective intermediate portions 611a, 611b. In the embodiment illustrated in FIG. 6A, the intermediate portions 611 are the narrowest portions of the I-shaped aperture 600, and the widths of the middle portion 610 and end portions 612 may be approximately the same. The width of the end portions 612 may also be less than the width of the middle portion 610 but greater than the narrowest portion of the intermediate portions 611. As shown in FIGS. 6B and 6C, apertures 600-2 and 600-n of different discrete sections 510 may define apertures that are similar in shape but have different sizes.

With reference to FIG. 6D, apertures 600 defined by one or more discrete sections 510 may be an aperture 600. may have a dimension (d1) or width of the bulbous center section 610 that is about 0.147″, a dimension (d2) or width of the narrowest intermediate portions 611a, 611b of about 0.0069″, and a dimension (d3) or width of the top and bottom or end portions 612a, 612b of about 0.0144″. The dimension (d4) or the height or length of the aperture 600 may be about 0.003″. The radius of curvature (r1) may be about 0.01″, and the radius of curvature (r2) may be about 0.0019″.

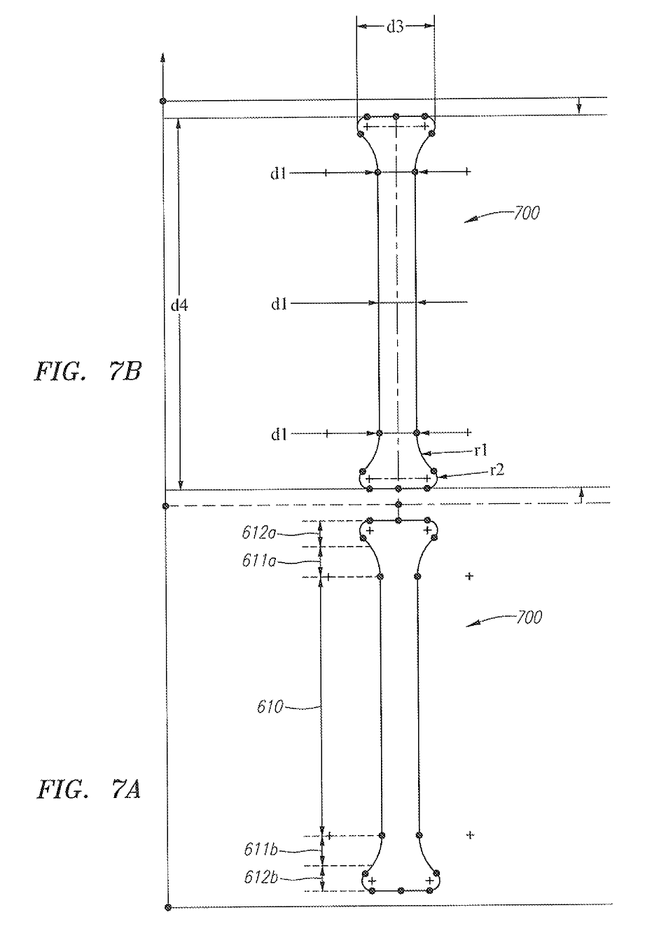

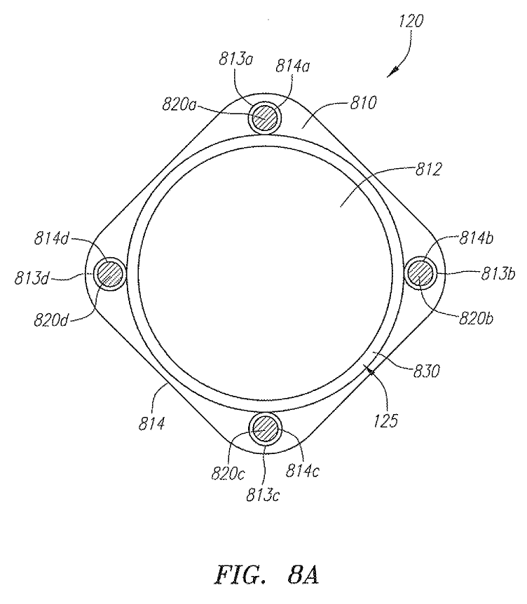

Other embodiments may utilize other symmetrical aperture configurations and formations of apertures. For example, referring to FIGS. 7A-B, in another embodiment, apertures 514a-n may be symmetrically shaped I-shaped apertures 700 having a middle portion 610 that is narrower than other portions of the aperture 700. Thus, the symmetrical I-shaped aperture 700 shown in FIGS. 7A-B has a “bone-like” shape. More particularly, in the illustrated embodiment, the middle portion 610 is the narrowest portion of the aperture 700, the intermediate portions 611a, 611b have larger widths than the middle portion 610, and the end portions 612a, 612b have larger widths than the middle portion 610 and the intermediate portions 611a, 611b.

With reference to FIG. 7B, which illustrates an aperture 700 that may, for example, be an aperture 514-1 defined by the discrete section 510-1 (or another aperture 514 defined by another discrete section 510 depending on the spine 430 configuration), the dimension (d1) or width of the center section 610 may be about 0.0069″, the dimension (d3) or width of the top and bottom or end portions 612a, 612b may be about 0.0144″, the dimension (d4) or the total height or length of an aperture may be about 0.003″, the radius of curvature (r1) may be about 0.01″, and the radius of curvature (r2) may be about 0.0019″. It should be understood that the dimensions described with reference to FIGS. 6A-C and 7A-B are provided only as examples, and that other dimensions may be utilized. I-shaped aperture 600, 700 dimensions may be adjusted and scaled accordingly.

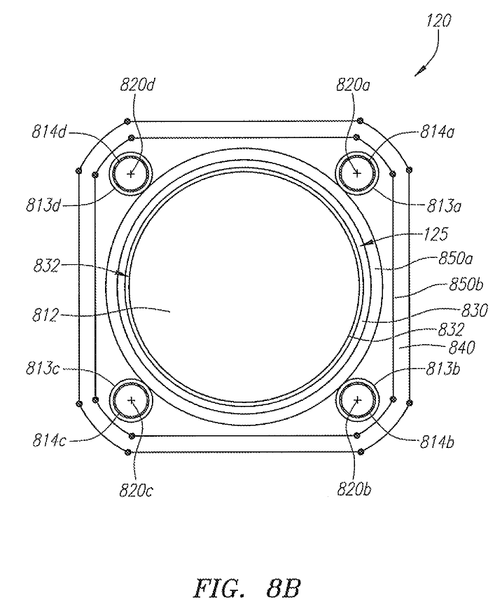

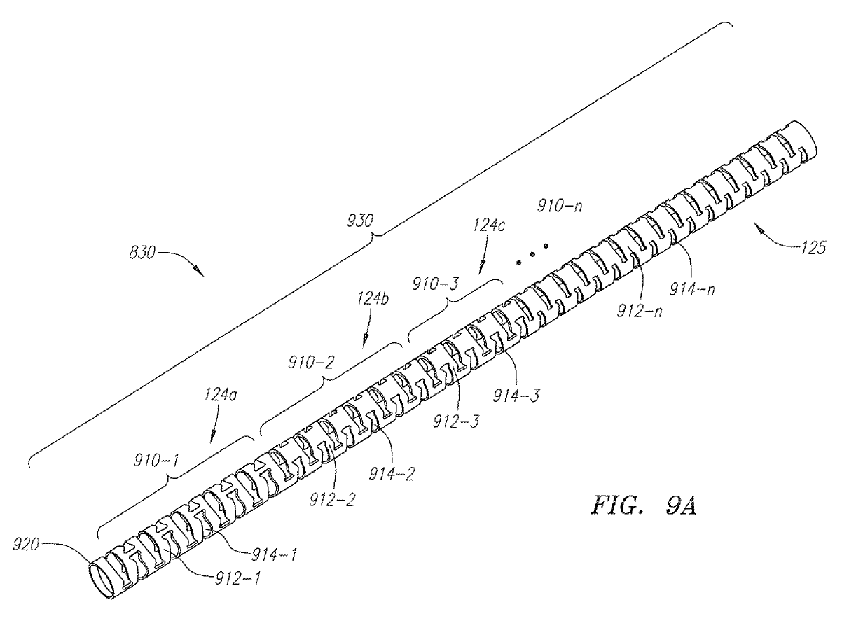

Referring to FIGS. 8A and 8B (which illustrates one manner in which the structure shown in FIG. 8A may be implemented in further detail), a catheter instrument 120 having a flexible and torquable apparatus, spine or support structure 830 (generally referred to as spine 830) may be constructed in a manner that is similar to the sheath instrument 110 described above with reference to FIGS. 4A-7B, with certain structural differences. In the embodiment illustrated in FIG. 8A, the elongate portion 122 of the catheter 120, which may have a length of about 88 cm, or about 35″, includes a body 810 that may be made of a polymeric material such as PTFE, PTFE doped polyimide, nylon, Pebax™, or another suitable material. The body 810 may be formed by a molding process or other suitable process. The polymeric material may be substantially flexible, such that it may be bent (up or down), steered (pitch or yaw), or rotated relatively freely without any substantial resistance or constraint to these movements.

The catheter body 810 defines a central lumen 812 that is configured to receive one or more working instruments 130, such as ablation catheters, guide wires, needles, scissors, clamps, etc. The central lumen 812 is configured such that these working instruments 130 may pass through the lumen 812 to the distal section of the elongate portion 122 of the catheter 120 and to the target site. For this purpose, the central lumen 812 may have an inner diameter or diagonal measurement of about 0.026″ (2 French) to about 0.041″ (slightly larger than 3 French).

In the illustrated embodiment, the outer surface of the elongate portion 122 of the catheter 120 is shaped to correspond to the “keyed” configuration of the central lumen 412 of the sheath 110. In the illustrated embodiment, the shape of the elongate portion 122 of the catheter 120 is a square shape with rounded corners (e.g., corresponding to the lumen 812 having rounded corners as shown in FIG. 4B), but other key shapes may be utilized such that the elongate portion 122 of the catheter 120 does not rotate or does so to a minimal degree within the central lumen 112 of the sheath 110. For this purpose, an elongate portion 122 of the catheter 120 may have an outer diameter or diagonal dimension of about 0.085″, e.g., for use with a central lumen 412 of the sheath body 410 that has an inner diameter or diagonal measurement of about 0.053″ (4 French) to about 0.092″ (7 French).

In the illustrated embodiment, the catheter body 810 includes a plurality of tubes 813a-d (generally 813) that define a plurality of smaller lumens 814a-d (generally 814) that are configured to receive or accommodate respective control elements or control wires 820a-d (generally 820). The catheter 120 may be structured such that the center-to-center distance between opposing lumens 814a and 814c, and 814b and 814b, is about 0.064″. The control wires 820 are manipulated by a corresponding splayer 220 to steer the distal section of the elongate portion 122 of the catheter 120 with pitch, yaw, and rotational movements. These maneuvers may be used to navigate the elongate portion 122) of the catheter 120 through tortuous natural body pathways (arteries, veins, etc.) in minimally invasive surgical procedures. For this purpose, a control wire 820 of the catheter 120 may have an outer diameter of about 0.0085″, and an inner diameter of a tube 813 that defines a control wire lumen 820 may have an inner diameter or width of about 0.010″ to about 0.012″ to accommodate a control wire 820.

The elongate portion 122 of the catheter 120 includes a flexible and torquable spine 830. In the illustrated embodiment, the catheter 120 is configured such that the spine 830 is positioned inwardly relative to tubes 813 and control element lumens 814. Thus, the spine 430 in certain embodiments of a sheath 110 surrounds these components, whereas these components surround the spine 830 in the illustrated embodiment of the catheter 120.

Certain figures illustrate an elongate portion 122 of a catheter 120 including a single spine 830, but in other embodiments, an elongate portion 122 may include multiple spines 830a-n (generally 830n). According to one embodiment, an elongate portion 122 of a sheath 120 includes multiple spines 830n that partially or completely overlap each other. According to another embodiment, a catheter 120 may include an elongate portion 122 having spines 830n that are arranged end-to-end. Further, in another embodiment, an elongate portion 122 of a catheter 120 may include certain spines 830n that partially or completely overlap, and other spines 830n that are arranged end-to-end. Accordingly, Figures that illustrate an elongate portion 122 of a catheter 120 that includes a single spine 830 are provided for ease of explanation and illustration, and it should be understood that other embodiments may involve a catheter 120 having multiple spines 830n, which may or may not be the same size, and which may or may not be overlapping. Further, elongate bodies of each spine 830n can define respective pluralities of discrete sections 910, and in a given plurality of discrete sections 910 1-n, at least one discrete section 910 is structurally different than other sections 910.

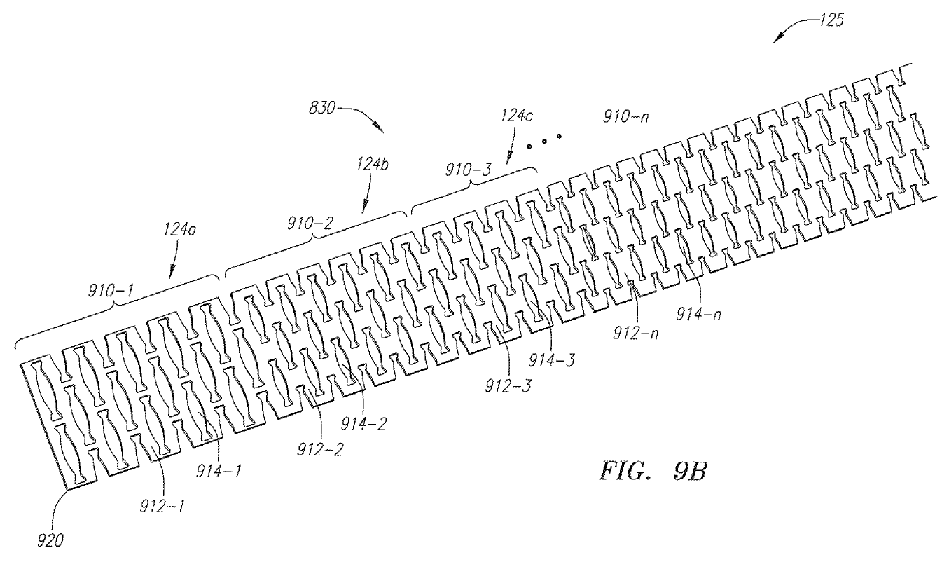

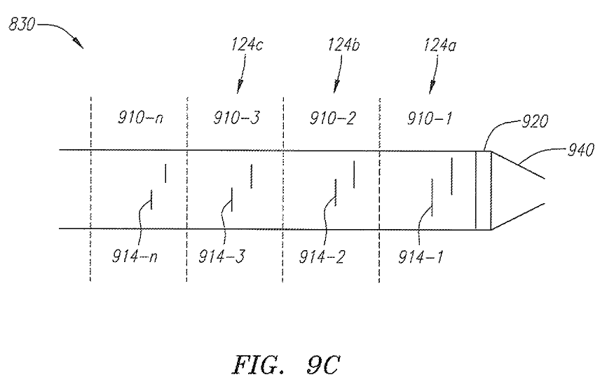

The elongate body of the spine 830 may extend along a portion (e.g., along a distal portion) or along the entire length of the elongate portion 122 of the sheath 120. The spine 830 may have a thickness of about 0.002″ such that the spine 830 has an inner diameter of about 0.042″, and an outer diameter of the spine 830 is about 0.046″. An inner lining 832 may also be applied to an inner surface of the spine 830 and may have a thickness of about 0.0005″ to about 0.002″, an inner diameter of about 0.040″ and an outer diameter of about 0.042″. Control wires 820 may be attached to a control ring (not shown in FIG. 8, but one example of which is illustrated in FIGS. 9-11) that is coupled to a distal portion of the spine 830. Alternatively, control wires 820 may be attached directly to distal portions of the spine 830.

The elongate portion 122 of the catheter 120 also includes an outer jacket or cover 840, which encases all of the aforementioned structures into a catheter unit or assembly 120. For this purpose, the cover 840 may be constructed, fabricated, or formed from a flexible and bio-compatible material for use in the human body. For example, the cover 840 may be formed from a polymeric material, e.g., a urethane material, poly-urethane material, nylon, Pebax™, etc. The cover 840 may have a thickness of about 0.007″, an inner diameter or diagonal measurement of about 0.078″ (about 6 French), and an outer diameter or diagonal measurement of about 0.092″ (about 7 French) such that the elongate portion 122 of the catheter 120 may slide along or through the central lumen 112 of the sheath 110 relatively freely or smoothly.

As illustrated in FIG. 8B, an inner braided layer 850a may be positioned between the spine 830 and tubes 813 that define control element lumens 814m and an outer braided layer 850b may be provided between the outer cover or jacket 840 and a tube 813. In this manner, the outer braided layer 850b may be a part of the jacket or cover 840. Such braided layers 850 may be used to alter bending, torque, axial (column strength), and radial (kink resistance) strength properties, and provide support for control wires 820 and tubes 813.

Referring to FIGS. 9A-C, one embodiment of a spine 830 for use in an elongate portion 122 of a catheter 120 includes an elongate body that is a unitary structure 930 including a plurality of discrete sections 910-1 to 910-n (generally referred to as discrete section 910). A control ring 920 may be attached to, or a part of, a distal most discrete section 910-1 (as shown in FIG. 9C), and control wires 820 may be attached to the control ring 920. A unitary structure 930 includes or is formed to have discrete sections 810 and has variable flexibility along its length while providing transmission of torque or twisting forces.

As shown in FIGS. 5A-C and 9A-C, according to one embodiment, spines 430, 830 for use in respective sheath and catheter instruments 110, 120 may be structured in the same or similar manner (but have different dimensions). In one embodiment, only the sheath 110 includes a spine 430. In another embodiment, only the catheter 120 includes a spine 830. In a further embodiment, the sheath 110 includes the spine 430, and the catheter 120 includes the spine 830, each spine having dimensions for use with respective instruments 110, 120. The spine 430 may be structured in the same manner as the spine 830 (as shown in FIGS. 5A-B and 9A-B), or the spines 430, 830 may have different geometric structures. In one embodiment, the spine 430 has a first geometric structure, and the spine 830 has a second geometric structure that is different than the first geometric structure. For ease of explanation, reference is made to the sheath 110 and the catheter 120 including respective spines 430, 830 that have the same or substantially similar structural configuration as shown in FIGS. 5A-B and 9A-B.

In the illustrated embodiment, each discrete section 910 includes segmental elements (912-1, 912-2, 912-3, . . . 912-n) (generally 912) between which are apertures, gaps or spaces (914-1, 914-2, 914-3, . . . , and 914-n) (generally 914) there between. Apertures 914 may be cut from material that forms a discrete section 912, or apertures 914 may be formed by cutting apertures 914 from unrolled or flat material that is later formed or rolled into the configuration as shown in FIG. 5 or utilizing other suitable fabrication methods. Referring to FIG. 9C, the spine 830 may include a soft distal tip 940 that may be constructed from a soft polymeric material to facilitate advancement through tortuous vasculature. In the illustrated embodiment, the soft distal tip 940 is attached to the control ring 920 or the distal tip of the spine 830 and the cover 840. Although FIGS. 9A-C illustrate three distinct discrete sections 510, other embodiments may include other numbers (n) of discrete sections 510, e.g., two, four, ten, twenty, and other numbers (n) of discrete sections 510. Thus, FIGS. 5A-B are provided as on example of how embodiments may be implemented.

Similar to the spine 430 for use in a sheath 110, a spine 830 for use in a catheter 120 includes a plurality of discrete sections 910 and has a tube-like shaped elongate body According to one embodiment, the elongate body of the spine 830 comprises a unitary structure 930 having a plurality of discrete sections 510, each of which has at least one distinguishing structural attribute that differentiates it from the other discrete sections 510. The arrangement of the discrete sections 510 that form the unitary structure 930 results in a torquable unitary structure 930 having variable flexibility that allows the elongate portion 122 of the catheter 120 to be positioned in its intended position as controlled by one or more control wires 820 and maintained in its intended position in the presence of external forces.

According to one embodiment, the flexibility of the spine 830 increases from a proximal portion to a distal portion such that the distal end, or discrete section 910-1, is the most flexible discrete section 910. In other embodiments, an intermediate section, e.g., one or more of discrete sections 910-2 and 910-3, may be stiffer than another discrete section 910 such that the flexibility varies along the length of the spine 930, but does not increase towards the distal most discrete section. For ease of explanation, reference is made generally to a spine 930 having flexibility that increases along its length towards to its distal end.

The apertures 914 defined by the spine 830 for use in an elongate portion 122 of the catheter 120 can also be I-shaped apertures as shown in FIGS. 9A-B, and as described with reference to FIGS. 5A-B and 6A-7B. The dimensions of the apertures 514 of the sheath spine 430 described with reference to FIGS. 6A-7B can, as necessary, be reduced or scaled for the catheter spine 830. Thus, details regarding suitable I-shaped apertures 914 and other embodiments are not repeated here.

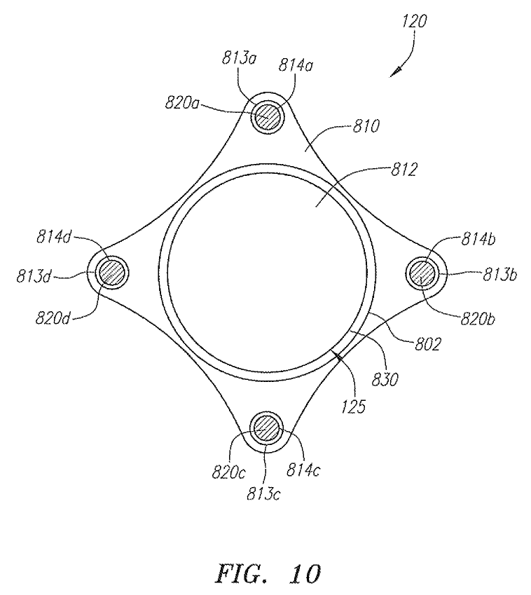

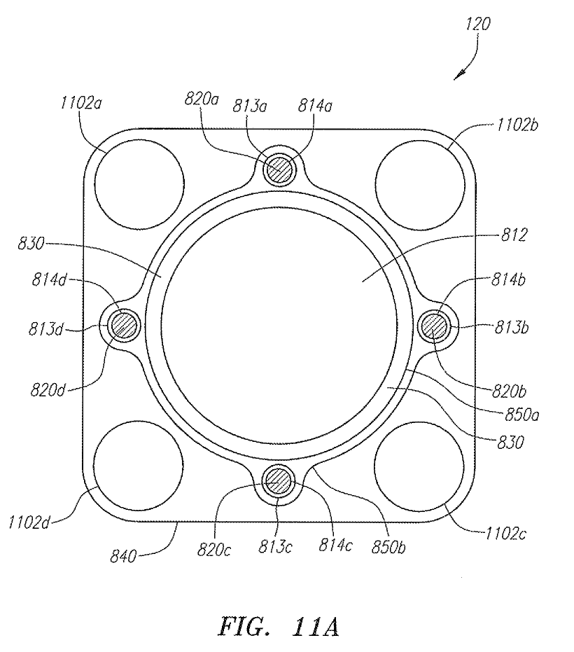

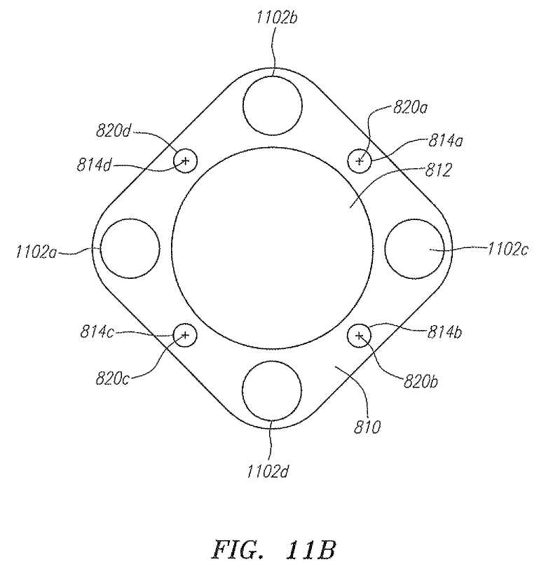

FIGS. 10 and 11A-B illustrate elongate portions 122 of catheters 120 constructed according to other embodiments and having different outer surface designs or key arrangements. In the embodiment illustrated in FIG. 10, the outer body 810, which may be made of a polymeric material such as Pebax™, for example, is shaped to have a different key surface and smaller profile compared to the configuration of the elongate portion 122 of the catheter 120 shown in FIGS. 8A-B. The spine 830 for use in a catheter 120 illustrated in FIG. 10 can be configured in the same or similar manner as described with reference to FIGS. 8A-9C, and apertures 914 within the spine 830 may also have a configuration that is the same as or substantially similar to apertures shown in FIGS. 6A-7B.

In the embodiment illustrated in FIGS. 11A-B, the catheter body 810 defines a square-like key shape and or more additional lumens (four additional lumens 1102a-d are illustrated) for delivery of other components that may be used for other purposes or in other applications. For example, one or more the lumens 1102a-d may be used to advance optical fibers (e.g., as a light source or for imaging), a laser, other tools, and flushing fluids, etc. to the distal portion of the elongate portion 122 of the catheter 122. For this purpose, the inner diameter or width of the lumens 1102a-d may be about 0.027″, e.g., for a body 810 defining a central or “basket” lumen 812 that may be used for advancement of working instruments 130 may have a diameter or width of about 0.42″. Structural configurations other than those illustrated in FIGS. 10 and 11A-B may also be utilized, e.g., depending on the manufacturing method utilized. For example, although the central and control element lumens are illustrated in FIGS. 11A-B as distinct lumens, the lumens may also be in communication with each other. Further, one or more braid layers, 850 may surround or be applied around one or more or all of the additional lumens 11102.

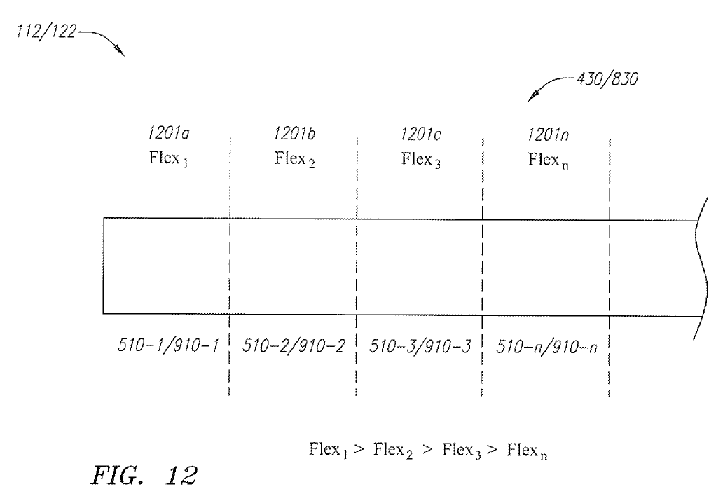

Having described embodiments of elongate portions 112, 122 of respective sheath and catheter instruments 110, 120, and the respective spine structures 430, 830 for use in such instruments 110, 120, further embodiments are described with reference to FIGS. 12-30, which generally illustrate an instrument having a spine 430, 830 or portion thereof that comprises discrete sections 510, 910. Embodiments may apply to only the elongate portion 112 of a sheath 110 (e.g., if the elongate portion 122 of the catheter 120 does not include a spine 830), only the elongate portion 122 of the catheter 120 (e.g., if the elongate portion 112 of the sheath 110 does not include a spine 430), or embodiments may apply to both (e.g., both elongate portions 112, 122 include respective spines 430,830). As such, FIGS. 12-30 refer to components of both sheath and catheter instruments 110, 120, noting that such embodiments may apply to a sheath 110 and/or a catheter 120. Further, it should be understood that although FIGS. 12-30 illustrate a spine having an elongate body that is a unitary structure defining a certain number (e.g., three) discrete sections for purposes of explanation and illustration, embodiments may also involve unitary structures having other numbers of discrete sections, as indicated by the “nth” discrete section. Further, although embodiments are described with reference to FIGS. 5A-B and 9A-B, embodiments may be implemented with other spines 430, 830 that include various structural configurations and other symmetrical aperture 514, 914 shapes.

Referring to FIG. 12, according to one embodiment, a spine structure 430, 830 is a unitary structure 530, 930 that includes or is formed to have discrete sections 510, 910 and a variable flexibility 1201a-n (Flex1-n) and torque transmission along its length such that the flexibility of the unitary structure 530, 930 increases from a proximal discrete section to a distal discrete section. Thus, in the illustrated embodiment, the discrete section 510-1, 910-1 is more flexible than the more proximal discrete sections, the discrete section 510-2, 910-2 is more flexible than the more proximal discrete sections, and so on, such that the discrete section 510-1, 910-1 is the most flexible discrete section, and the corresponding part of the elongate portion 112, 122 is the most flexible portion.

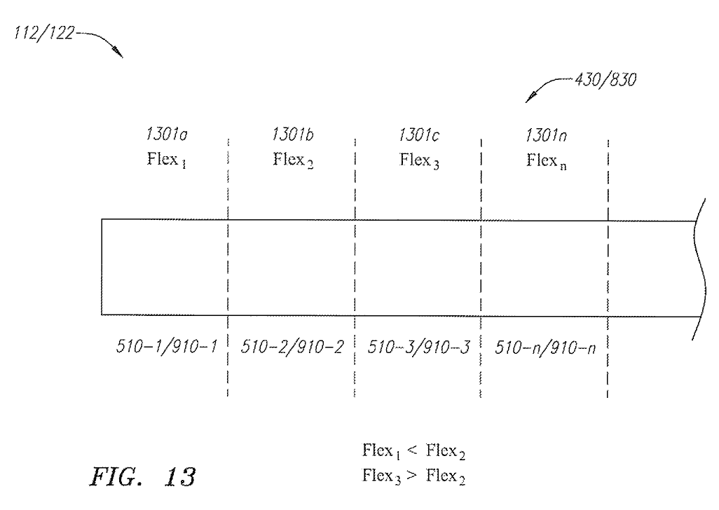

Referring to FIG. 13, in another embodiment, a spine structure 430, 830 is a unitary structure 530, 930 that includes or is formed to have discrete sections 510, 910 that have different flexibilities 1301a-n (Flex1-n) such that the flexibility varies along the length of the unitary structure 530, 930 while also providing for torque transmission, but the flexibility may not increase from the proximal most section to the distal end since an intermediate distal section, e.g., section 510-2, 910-2, may be stiffer than the distal most discrete section 510-1, 910-1. Other embodiments may involve other flexibility variations and arrangements of discrete sections 510, 910. As described in further detail below, flexibility variations can be controlled, selected or customized by one or more or all of the discrete sections 510, 910 having one or multiple distinguishing attributes relative to one or multiple other discrete sections 510, 910.

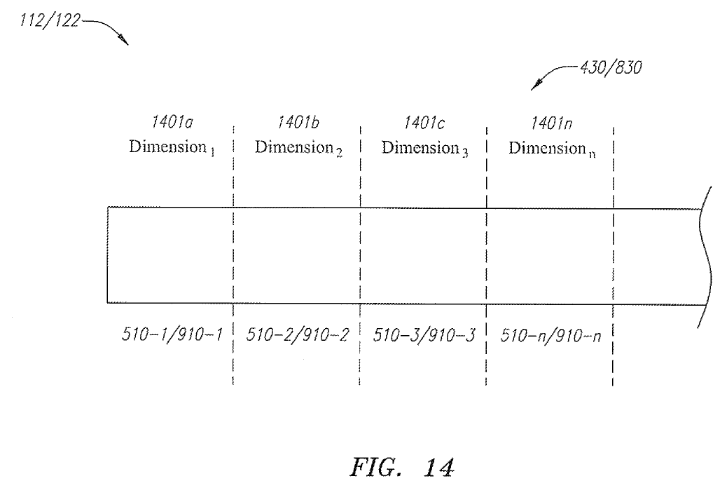

Referring to FIG. 14, one manner in which embodiments may be implemented is by varying the geometric structure of the elongate portion 112, 122, e.g., by varying one or more dimensions 1401a-n (Dimensions 1-n) of a discrete section 510, 910 relative to one or more other discrete sections 510, 910. Embodiments may involve varying one or multiple dimensions in one or more or all of the discrete sections 510, 910

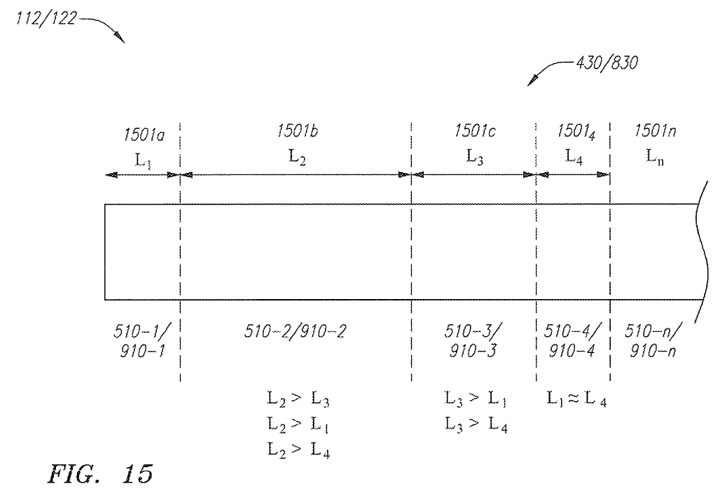

For example, referring to FIG. 15, according to one embodiment, variable flexibility while providing for torque transmission is achieved by at least one discrete section 510, 910 having a different length 1501a-n (L1-Ln) than other discrete sections 510, 910. Such embodiments may be utilized in cases in which, for example, discrete sections 510, 910 are of the same material and have other dimensions that are the same or substantially similar. Embodiments may involve one, multiple, or all of the discrete sections 510, 910 being distinguished on this basis. In other embodiments, two or more discrete sections 510, 910 may have the same length but another distinguishing attribute.

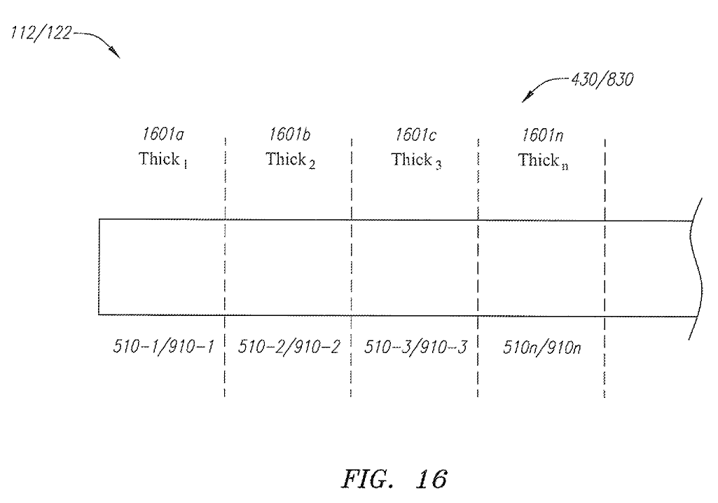

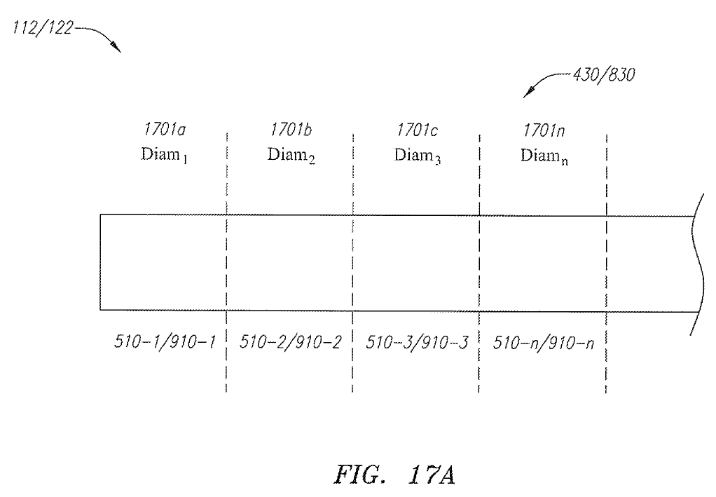

Referring to FIG. 16, according to another embodiment, a spine 430, 830 having variable flexibility and torque transmission includes at least one discrete section 510, 910 having different geometric attributes compared to other discrete sections 510, 910 based on being formed of materials of different thicknesses 1601a-n (Thick1-n). Such distinguishing attributes may be utilized in cases in which, for example, discrete sections 510, 910 are of the same material and have other dimensions that are the same or substantially similar. Embodiments may involve one, multiple, or all of the discrete sections 510, 910 being distinguished on this basis. In other embodiments, two or more discrete sections 510, 910 may have the same thickness but are distinguished based on another structural attribute.

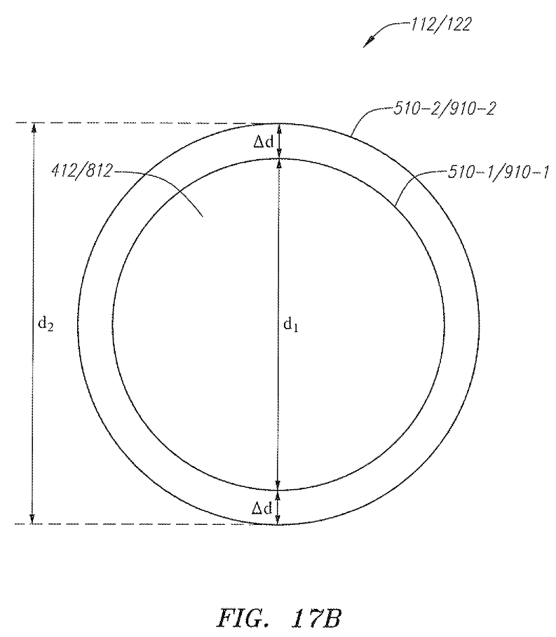

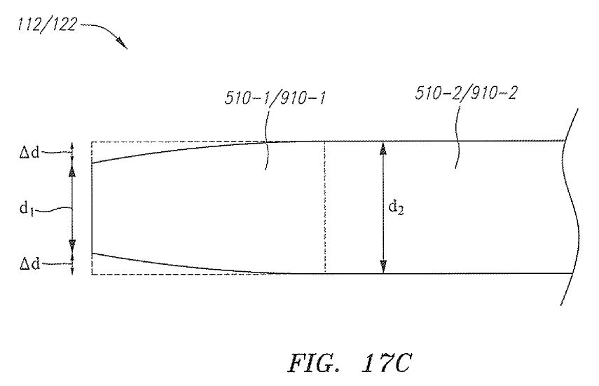

Referring to FIG. 17A, according to another embodiment, a spine 430, 830 having variable flexibility and torque transmission is achieved by at least one discrete section 510, 910 having different geometric attributes in the form of different widths or diameters 1701a-n (Diam1-n) than other sections 510, 910. Such distinguishing attributes may be utilized in cases in which, for example, discrete sections 510, 910 are of the same material and have other dimensions that are the same or substantially similar.

For example, referring to FIGS. 17B-C, in one embodiment, the distal segment 510-1, 910-1 may taper from a width or diameter (d2), which may be the width or diameter of a proximal segment 510-2, 910-2, to a smaller width or diameter (d1). According to one embodiment, the smaller diameter (d1) may be about 15-20% less, e.g., about 17% less, than the larger diameter (d2). For example, the larger diameter (d2) may be about 0.158″, the smaller diameter (d1) may be about 0.131″, and Δd may be about 0.0135. Other embodiments may involve other tapering ratios and dimensions. Embodiments may involve one, multiple, or all of the discrete sections 510, 910 being distinguished on this basis. In other embodiments, two or more discrete sections 510, 910 may have the same diameter but are distinguished based on another structural attribute.

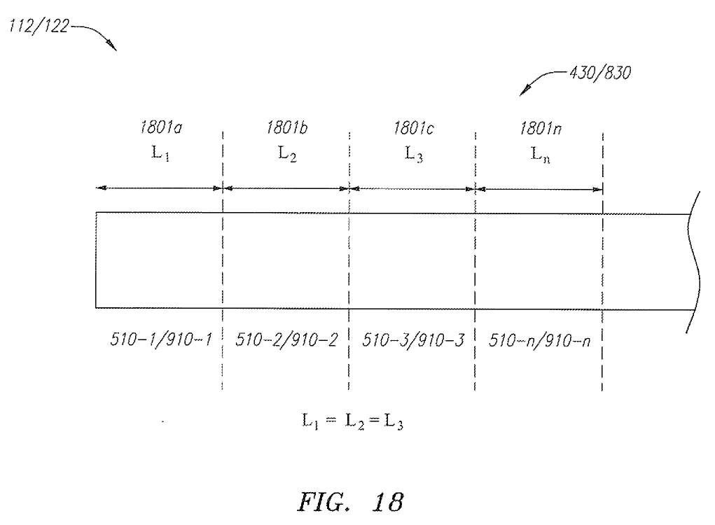

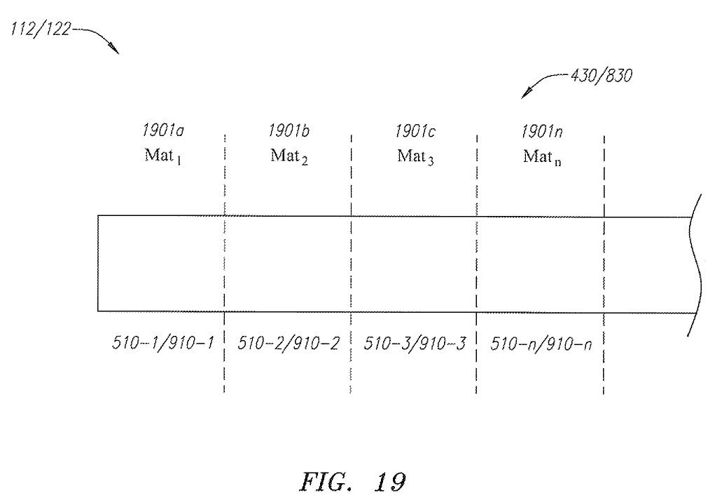

Referring to FIG. 18, according to another embodiment, a spine 430, 830 having variable flexibility and torque transmission includes two or more or all of the discrete sections 510, 910 having the same or substantially the same lengths 1801a-n (L1-n), but one or more other distinguishing attributes, such as being made of different materials, having different widths and/or different thicknesses, that contribute to variable flexibility.

For example, in another embodiment, referring to FIG. 19, variable flexibility while providing for torque transmission if achieved by at least one discrete section 510, 910 being made of different material 1901a-n or having a different material attribute (generally referred to as Mat1-n) than other discrete sections 510, 910. According to one embodiment, different sections 510, 910 are made of different materials, which may be more flexible materials such as a high spring constant stainless steel, a high spring constant nitinol, etc., and less flexible materials such as lower spring constant stainless steel, lower spring constant nitinol, etc.). Because of these differences, one discrete section (e.g., distal section 510-1, 910-1 as shown in FIG. 12) may have greater flexibility than more proximal sections 510, 910 for greater degrees of bending, steering, and rotation than more proximal sections 510, 910. Embodiments may involve one, multiple, or all of the discrete sections 510, 910 being distinguished on this basis. Further, certain sections 510, 910 may be made of the same material but have another distinguishing attribute, such as thickness, diameter, etc., which contributes to variable flexibility.

In another embodiment, a discrete section 510, 910 may be structurally distinguished from other discrete sections 510, 910 based a density of material or other different material properties. For example, variable flexibility is the result of a discrete section 510, 910 having different braid 850 densities, i.e., different numbers of braid 850 segments per length. For example, with reference to FIG. 4B, the densities of braids 450, 850 may be changed to define a discrete section 510. As another example, the densities of braids 450, 850 may be changed to define a discrete section 910. A discrete section 510, 910 having a higher braid density may be stiffer than a discrete section 510, 910 having a lower braid density.

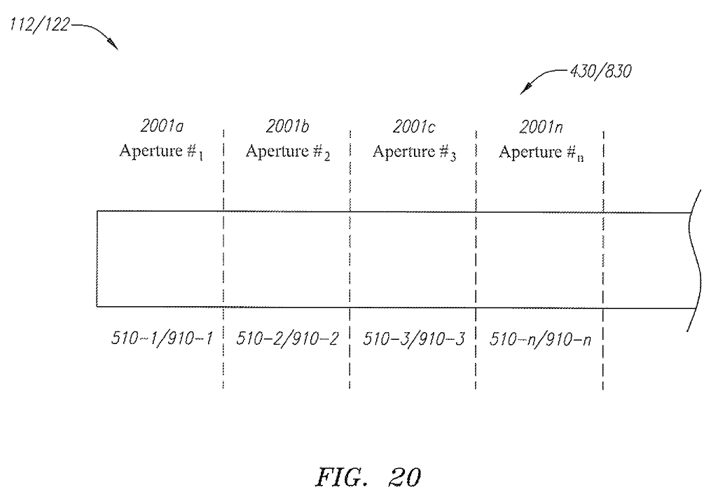

Referring to FIG. 20, according to another embodiment, variable flexibility while providing for torque transmission is controlled or customized based on the number 2001a-n(Aperture#1-n) of apertures 514 defined by each discrete section 510, 910, at least one discrete section 510, 910 having a different number of apertures 514, 914 than other sections 510, 910 of the elongate portion 112, 122. Embodiments may involve one, multiple, or all of the discrete sections 510, 910 being distinguished on this basis. Further, certain sections 510, 910 that have the same number of apertures 514, 914 may be distinguished based on another distinguishing attribute, such as material, thickness, diameter, etc., and other aperture attributes, as discussed below.

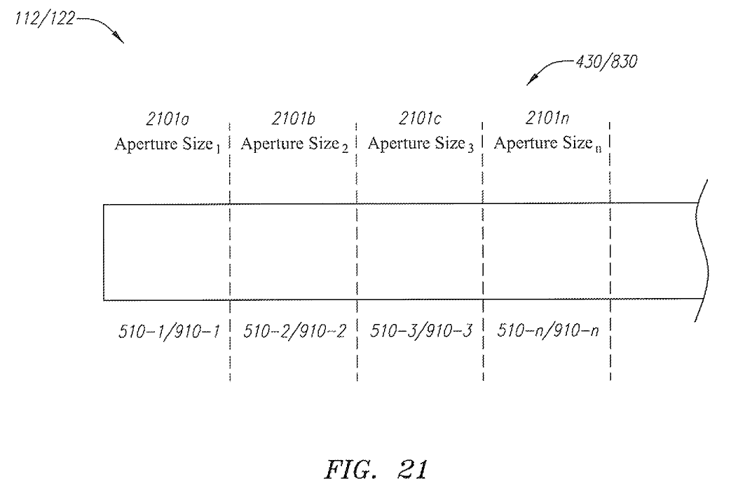

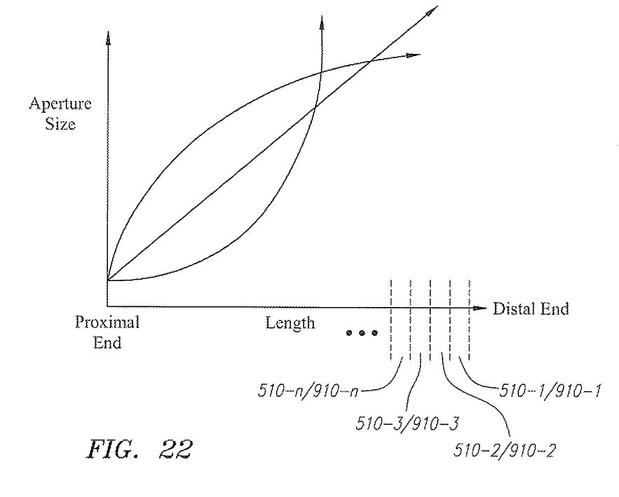

Referring to FIG. 21, variable flexibility while providing for torque transmission may also be achieved and controlled or customized based on at least one discrete section 510, 910 having apertures 514, 914 that are a different size (2101a-n) (ApertureSize1-n) compared to apertures 514, 914 of other sections 510, 910. For example, as shown in FIGS. 5, 6A-C, and 9A, one discrete section, such as the distal discrete section 510-1, may include apertures 514-1 that are larger than apertures of other discrete sections 514. One or more or all of the discrete sections 510, 910 may be distinguished on this basis.

Referring to FIG. 22, in one embodiment, the aperture 514, 914 size increases from a proximal portion to a distal portion of the elongate portion 112, 122 of sheath and/or catheter instruments 110/120, and FIG. 22 graphically illustrates different manners in which aperture 514, 914 size may vary. According to one embodiment, aperture 514, 914 size varies linearly with length, e.g., linearly with each discrete section 510, 910, which may include a one or multiple apertures 514, 914. According to another embodiment, aperture 514, 914 size varies non-linearly, e.g., exponentially, with length. Such embodiments may involve aperture 514, 914 size varying non-linearly with each discrete section 510, 910 or over multiple discrete sections 510, 910, which may include a single aperture or multiple apertures. Embodiments may involve one, multiple, or all of the discrete sections 510, 910 being distinguished on this basis. Further, certain sections 510, 910 having apertures 514, 914 that are the same size may be distinguished based on another distinguishing attribute, such as material, thickness, diameter, etc., and other aperture attributes

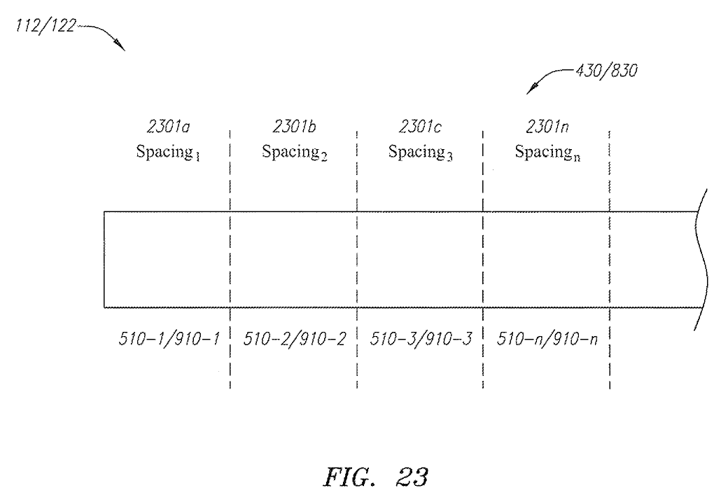

Referring to FIG. 23, variable flexibility may also be controlled or customized based on the spacing (2301a-n) (Spacing1-n) of the apertures 514, 914 defined by at least one discrete section 510, 910. Aperture spacing may relate to, for example, the number, shapes and/or sizes of apertures 514, 914. One or more or all of the discrete sections 510, 910 may be structurally distinguished on this basis, and other discrete sections 510, 910 that include apertures 514, 914 having consistent spacing may be distinguished based on other attributes.

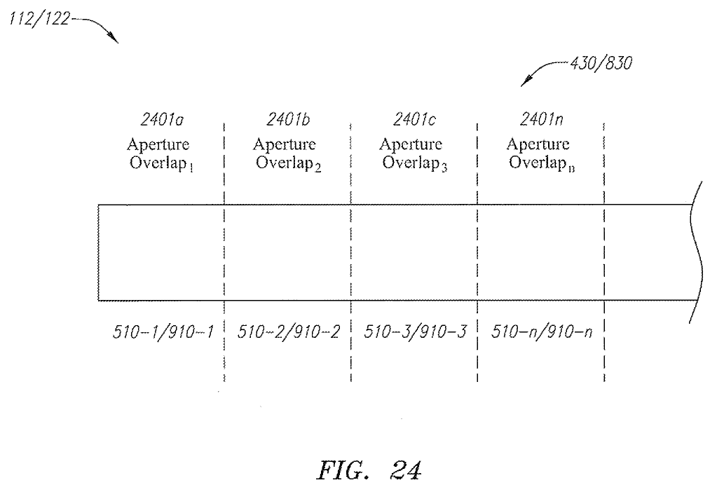

Referring to FIG. 24, according to another embodiment, variable flexibility may also be controlled or customized based on the degree of overlap (2401a-n) (ApertureOverlap1-n) of the apertures 514, 914 defined by at least one discrete section 510, 910. The degree of overlap may depend, in part, on the length or the degree to which an aperture subtends a discrete section 510, 910, the position of apertures 514, 914 within a discrete section 510, 910, or a combination thereof. One or more or all of the discrete sections 510, 910 may be structurally distinguished on this basis, and other discrete sections 510, 910 that include apertures 514, 914 having consistent spacing may be distinguished based on other attributes.

For example, as shown in FIGS. 5A-B, 6A-C, and 9A-B, and as generally illustrated in FIGS. 5C and 9C, apertures 514-1 within a distal discrete section 510-1 overlap each other to a greater degree than apertures 514-2 within distal discrete sections 510-1 to 510-n, and apertures 514-2 within discrete section 510-2 overlap each other to a greater degree than more proximal discrete sections. According to one embodiment, the degree of overlap may vary by about 0% (no overlap or a very small degree of overlap) to about 50% or more depending on the configuration of the apertures. Referring to FIGS. 5A-B, there is about a 50% overlap between apertures 514-1 in the distal discrete section 510-1, and about 33% overlap between apertures 514-2 in the discrete section 510-2, and less overlap, e.g., about 5-10% overlap, between apertures 514-n in discrete section 510-n. Different degrees of overlap may result from apertures 514, 914 subtending different angles such that the first discrete section 510-1 is more flexible than the second discrete section 510-2. Such apertures may overlap each other by different degrees, thereby resulting in variable flexibility along the length of an elongate portion 112, 122 of a sheath or catheter instrument 110, 120. One or more or all of the discrete sections 510, 910 may be structurally distinguished on this basis, and other discrete sections 510, 910 that include the same degree of apertures 514, 914 overlap may be distinguished based on other attributes. Further, discrete sections 510, 910 can be distinguished based on different shapes of apertures 514, 914 (e.g., one or more discrete sections 510, 910 may have I-shaped apertures as shown in FIGS. 5A-B and 9A-B, and other discrete sections 510, 910 may have I-shaped apertures 514, 914 as shown in FIGS. 7A-B, or another symmetrical shape.

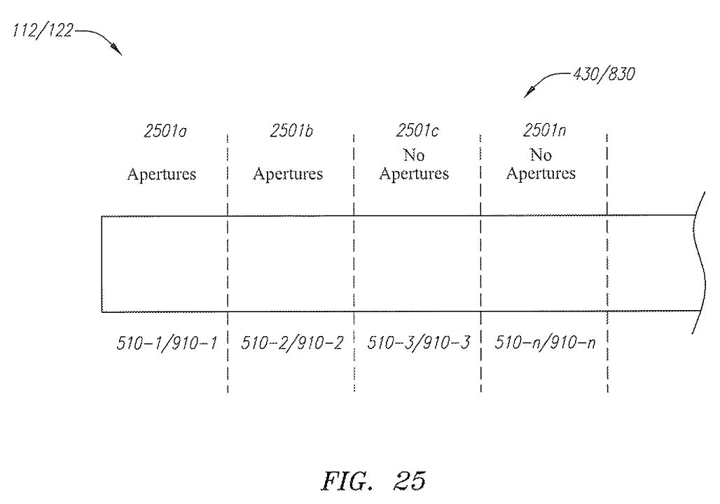

Referring to FIG. 25, although certain embodiments are described with reference to each distal section having apertures 514, 914, variable flexibility while providing for torque transmission may also be based on the presence of apertures, i.e., certain sections 510, 910 having apertures 514, 914 (as discussed above) whereas other discrete sections 510, 910 do not. In the illustrated embodiment, the distal most discrete sections 510-1, 910-1 may define apertures (2501a-b), whereas more proximal discrete sections may not (2501c-n). Other embodiments may involve different numbers and configurations of sections that have and that lack apertures. For example, rather than grouping together discrete sections 510, 910 that have and that do not have apertures 514, 914, other embodiments may have an interspersed or alternating pattern of discrete sections 510, 910 that have and that do not have apertures 514, 914. Thus, FIG. 25 is provided to generally illustrate one example of how flexibility can vary depending on the presence of apertures 514. One or more or all of the discrete sections 510, 910 may be structurally distinguished on this basis. Discrete sections 510, 910 that have apertures 514, 914 may themselves be distinguished from one or more other discrete sections 510, 910 based on other distinguishing attributes, e.g., number of apertures, aperture size, overlap, etc. Similarly, discrete sections 510, 910 that do not have apertures 514, 914 may themselves be distinguished from one or more other discrete sections 510, 910 based on other distinguishing attributes.

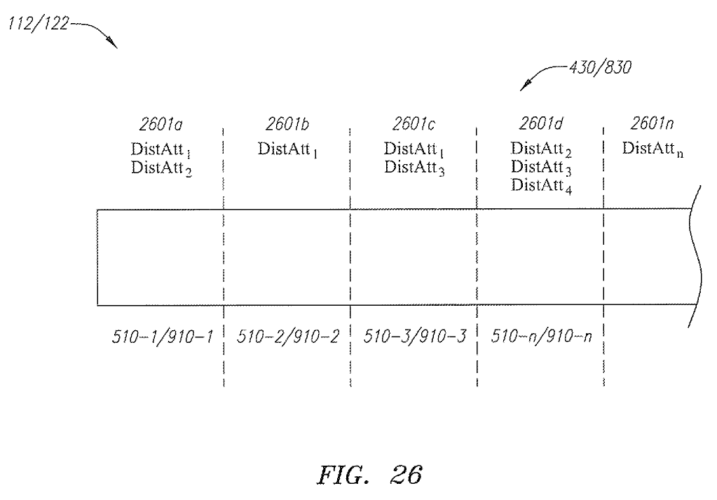

While certain embodiments are described with reference to a particular distinguishing attribute that results in one or more or all of the discrete sections 510, 910 being structurally distinguished in some manner, in other embodiments, a given discrete section 510, 910 may include multiple attributes that structurally distinguish that discrete section 510, 910 from other sections 510, 910. As generally illustrated in FIG. 26, a given discrete section 510, 910 may be distinguished from other sections 510/910 based on one, two, three and other numbers and various combinations of distinguishing attributes 2601a-n (DistAtt1-n).

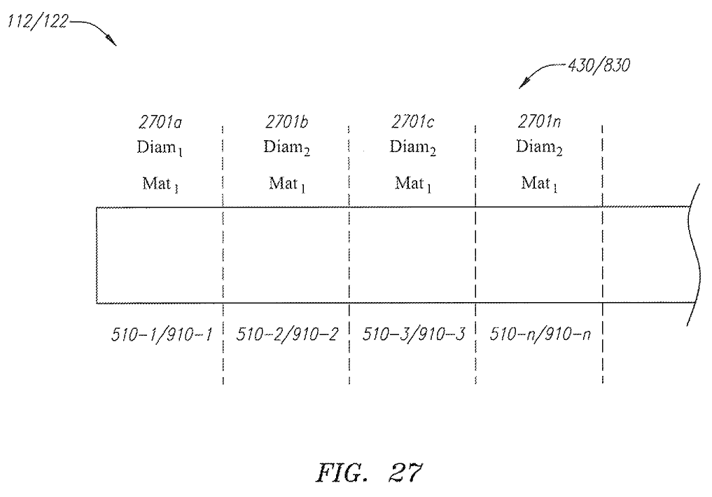

For example, in one embodiment illustrated in FIG. 27, combinations 2701a-n of the diameter and material of one or more discrete sections 510, 910 may be distinguished from other sections 510, 910 (e.g., as described with reference to FIGS. 17A-B and 19). One or more or all of the discrete sections 510, 910 may be structurally distinguished on this basis. Discrete sections 510, 910 that have the same diameter and that are made of the same material may themselves be distinguished from other discrete sections 510, 910 based on other distinguishing attributes. Thus, FIG. 27 is provided to generally illustrate one example of how flexibility can vary depending on a combination of distinguishing structural attributes.

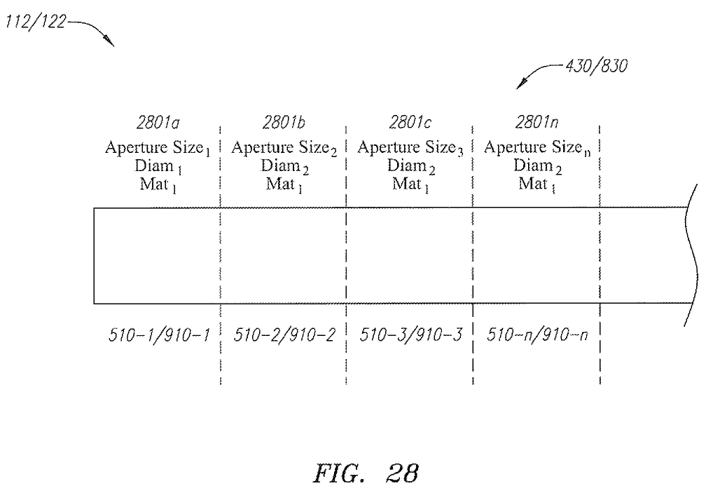

In another embodiment, referring to FIG. 28, one or more discrete sections 510, 910 may be distinguished from other sections 510, 910 as a result of having a different diameter, being made of a different material and having different aperture sizes 2801a-n (e.g., as described with reference to FIGS. 5A-B, 9A-B, 17A-B, 19, 21 and 22). One or more or all of the discrete sections 510, 910 may be structurally distinguished on this basis. Other discrete sections 510, 910 that have the same diameter, are made of the same material, and have aperture that are the same size may be distinguished on other bases. Thus, FIG. 28 is provided to generally illustrate another example of how flexibility can vary depending on a combination of distinguishing attributes.

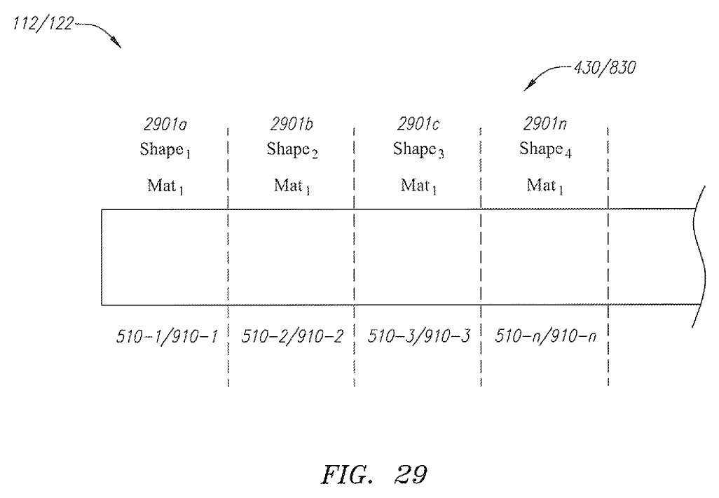

In a further embodiment, referring to FIG. 29, one or more discrete sections 510, 910 may be distinguished from other sections 510, 910 as a result of the combinations 2901a-n of apertures 514, 914 having a different shapes and discrete sections 510, 910 being made of different materials (e.g., as described with reference to FIGS. 5A-B, 6A-D, 7A-B, 9A-B and 19). One or more or all of the discrete sections 510, 910 may be structurally distinguished on this basis. Other discrete sections 510, 910 that have the same diameter, are made of the same material and have apertures 514, 914 that are of the same size may be distinguished on other bases. Thus, FIG. 29 is provided to generally illustrate a further example of how flexibility can vary depending on a combination of distinguishing attributes.

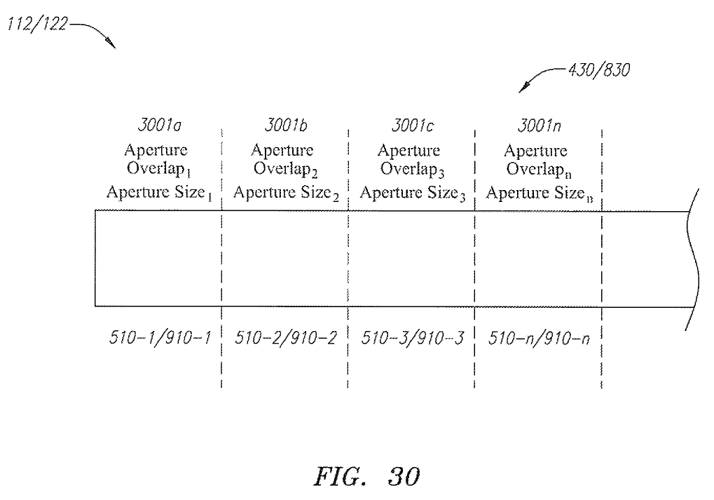

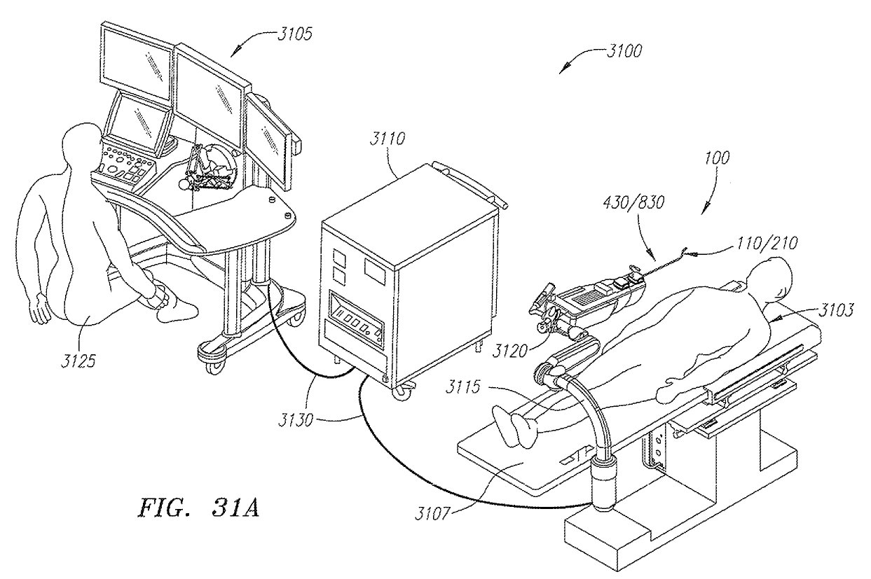

Referring to FIG. 30, in a further embodiment, one or more discrete sections 510, 910 may be distinguished from other sections 510, 910 as a result of having a different aperture 514, 914 sizes (e.g., as shown in FIGS. 5A-B, 9A-B and 21) and different degrees of aperture 514, 914 overlap (e.g., as shown in FIGS. 5A-B, 9A-B, and 24). Other discrete sections 510, 910 that have the same aperture sizes and overlap may be distinguished based on, e.g., one or more of the number of apertures, aperture shape (e.g., whether an I-shaped aperture has a middle portion that protrudes outwardly or is narrow than other portions), material thickness, diameter, and other distinguishing attributes. Thus, FIG. 30 is provided to generally illustrate another example of how flexibility can vary depending on a combination of distinguishing attributes.

Other embodiments may involve one or more or all of the discrete sections 510, 910 being distinguished based on one, two, three, four, five and other numbers of distinguishing attributes and different combinations thereof. Further, it should also be understood that distinguishing attributes can be used to differentiate discrete sections that are of the same length or that are of different lengths. Thus, for example, the number of apertures may differ in each discrete section having the same or different length, the aperture shape, size, and/or degree of overlap may different in each discrete section having the same or different length, etc.

FIGS. 31A-N and 32A-G illustrate robotic surgical systems in which embodiments may be implemented or with which embodiments may be utilized, and applications thereof. Referring to FIGS. 31A-B, one example of a robotic surgical system 3100 in which embodiments that utilize a sheath instrument 110 and/or a catheter instrument 120 having a spine 430, 830 includes an operator work or control station 3105, which may be configured as, or include, control, processor or computer software and/or hardware. The workstation 3105 is located remotely from an operating table 3107, an electronics rack 3110, a setup joint mounting brace 3115, and motor-driven controller in the form an instrument driver 3120. A surgeon or operator 3125 seated at the operator workstation 3105 monitors a surgical procedure, patient 3103 vitals, and controls one or more flexible catheter assemblies 100 that may include a coaxially-associated instruments of an outer sheath instrument 110 and an inner coaxially-associated catheter 120, such as a guide catheter (e.g., as described with reference to FIGS. 1-3). A working instrument 130, such as guidewire, a pusher wire, an ablation catheter, a laser ablation fiber, a grasper, a collapsible basket tool, etc., may be positioned within the working lumen 812 defined by the catheter 120 or coupled to or advanced by a distal end of the catheter 120.

Although the various components of the system 3100 are illustrated in close proximity to each other, components may also be separated from each other, e.g., in separate rooms. For example, the instrument driver 3120, the operating table 3107 and a bedside electronics box may be located in the surgical area, whereas the operator workstation 3105 and the electronics rack 3110 may be located outside of the surgical area behind a shielded partition. System 3100 components may communicate with other components via a network, thus allowing for remote surgery such that the surgeon 3125 may be in the same or different building or hospital site. For this purpose, a communication link or cables 3130 may be provided to transfer data between the operator control station 3105 and the instrument driver 3120. Wireless communications may also be utilized.

An example of a setup joint, instrument mounting brace or support assembly 3115 (generally referred to as a support assembly 3115) that supports the instrument driver 3120 above the operating table 3107 is an arcuate-shaped structure configured to position the instrument driver 3120 above a patient 3103 lying on the table 3107 for convenient access to desired locations relative to the patient 3103. The support assembly 3115 may also be configured to lock the instrument driver 3120 into position. In this example, the support assembly 3115 is mounted to the edge of a patient bed 3107 such that an assembly 100 including a catheter 120 mounted on the instrument driver 3120 can be positioned for insertion into a patient 3103 and to allow for any necessary movement of the instrument driver 3120 in order to maneuver the catheter assembly 100 during a surgical procedure. Although certain figures illustrate one instrument driver 3120, other systems may involve multiple instrument drivers 3120 attached to a single support assembly 3115.

Referring to FIG. 31C, one suitable operator workstation 3105 includes a console having one or more display screens 3132, a master input device (MID) 3134 and other components such as a touchscreen user interface 3136, and data glove input devices 3138. The MID 3134 may be a multi-degree-of-freedom device that includes multiple joints and associated encoders. MID 3134 software may be a proprietary module packaged with an off-the-shelf master input device system, such as the Phantom® from SensAble Technologies, Inc., which is configured to communicate with the Phantom® Haptic Device hardware at a relatively high frequency as prescribed by the manufacturer. Other suitable MIDs 3134 are available from suppliers such as Force Dimension of Lausanne, Switzerland. The MID 3134 may also have haptics capability to facilitate feedback to the operator, and software modules pertinent to such functionality may be operated on the master computer. An example of data glove software 3144 is a device driver or software model such as a driver for the 5DT Data Glove. In other embodiments, software support for the data glove master input device is provided through application drivers such as Kaydara MOCAP, Discreet 3D Studio Max, Alias Maya, and SoftImage|XSI.

The instrument driver 3120 and associated flexible catheter assembly 100 and working instruments 130 may be controlled by an operator 3125 via the manipulation of the MID 3134, data gloves 3138, or a combination of thereof. During use, the operator 3125 manipulates a pendant and MID 3134 to cause the instrument driver 3120 to remotely control flexible catheters that are mounted thereon. Inputs to the operator workstation 3105 to control the flexible catheter assembly 100 can entered using the MID 3134 and one or more data gloves 3138. The MID 3134 and data gloves 3138, which may be wireless, serve as user interfaces through which the operator 3125 may control the operation of the instrument driver 3120 and any instruments attached thereto. It should be understood that while an operator 3125 may robotically control one or more flexible catheter devices via an inputs device, a computer or other controller of the robotic catheter system 3100 may be activated to automatically position a catheter instrument 120 and/or the distal portion thereof inside of a patient or to automatically navigate the patient anatomy to a designated surgical site or region of interest.

Referring to FIG. 31D, a system architecture of one robotic catheter system 3100 in which embodiments may be implemented or with which embodiments may be utilized includes a controller in the form of a master computer 3141 that manages operation of the system 3100. The master computer 3141 is coupled to receive user input from hardware input devices such as a data glove input device 3138 and a haptic MID 3134. The master computer 3141 may execute MID hardware or software 3143, data glove software 3144 and other software such as visualization software, instrument localization software, and software to interface with operator control station buttons and/or switches. Data glove software 3144 processes data from the data glove input device 3138, and MID hardware/software 3143 processes data from the haptic MID 3134. In response to the processed inputs, the master computer 3141 processes instructions to instrument driver computer 3142 to activate the appropriate mechanical response from the associated motors and mechanical components of the driver 3120 to achieve the desired response from the flexible catheter assembly 100 including a sheath 110 and catheter 120.

As shown in FIGS. 1-3 and 31E-N, a flexible catheter assembly 100 for use in embodiments includes three coaxially-associated instruments including an outer sheath 110, an inner coaxially-associated catheter or guide catheter 120, and a working instrument 130 such as a stent, a guidewire, pusher wire, ablation catheter, laser ablation fiber, grasper, collapsible basket tool, etc., which is advanced through the working lumen 812 defined by the catheter 120.

In the illustrated example, the splayer 220 for the catheter 120 is located proximally of the splayer 210 for the sheath 210, each of which may include one or more control elements or pull wires. Both splayers 210, 220 are mounted to respective mounting plates on the instrument driver 3120, which controllably actuates one or more motors in the splayers 210, 220 are controlled to controllably manipulate the associated sheath and catheter instruments 110, 120. Such manipulation may involve rotation (FIG. 31J), pitch (FIG. 31K), yaw (FIG. 31L), multi- or omni-directional manipulation, e.g., a combination of rotation, pitch and yaw (FIG. 31M) and pitch and yaw (FIG. 31N) relative to axes 3151, 3152, 3153.

FIGS. 3 and 32A-G illustrate how the elongate portions 112, 122 of the assembly 100 is advanced through vasculature 3160 such as cardiac veins and arteries including the coronary sinus, carotid artery, etc., and navigated towards a target site where a working instrument 130 can be deployed or delivered. With embodiments that include a flexible and torquable spine 430, 830, the assembly 100 including the sheath 110 and catheter 120 may be manipulated, positioned and navigated through tortuous vasculature, during which the assembly may be configured as shown in FIGS. 32F and 32G.

Referring to FIG. 32F, the distal section of the elongate portion 122 of the catheter 120 including a spine 830 may be articulated to form arc with a substantially constant radius of curvature. Due to the variable flexibility of the spine 830 and corresponding section of the elongated portion 122, the tip of the elongate portion 122 of the catheter 120, the distal tip 111 of the elongate portion 112 of the sheath 110 may be used as a fulcrum or support base such that the elongate portion 122 of the catheter 120 can be controlled using that distal tip 111 support base to assume the shape of an arc having a substantially constant radius of curvature. FIG. 32G further illustrates now the distal portion of the elongate portion 122 of the catheter 120 can be bent into a “J” shape, which is particularly advantageous when navigating vasculature that includes sharp turns and angles.

Thus, embodiments of the invention that utilize a flexible and torquable spines 430 and/or 830 allow relatively short segments of assembly 100 components, such as the distal portion of the elongate portion 122 of the catheter, to be controllably articulated in a precise manner.

Although particular embodiments have been shown and described, it should be understood that the above discussion is not intended to limit the scope of these embodiments. While embodiments and variations of the many aspects of the invention have been disclosed and described herein, such disclosure is provided for purposes of explanation and illustration only. Many combinations and permutations of the disclosed embodiments are useful in minimally invasive surgery, and the system is configured to be flexible for use with other system components and in other applications. Thus, various changes and modifications may be made without departing from the scope of the claims.

For example, although particular examples of symmetrical I-shaped apertures are shown and described, other symmetrical apertures and other I-shaped apertures may be utilized. For example, other I-shaped apertures may, for example, have even larger or narrower middle portions, wider or narrower end portions, and other dimensional variations. Accordingly, the symmetrical apertures shown in various figures are provided in a non-limiting manner to illustrate examples of how embodiments may be implemented.

Further, embodiments may be utilized with sheath and catheter instruments that are made of different materials and that have different dimensions. Thus, the dimensions provided in this specification are provided in a non-limiting manner as examples of how embodiments may be implemented. Further, various system components including catheter components may be made with materials and techniques. It should be understood that one or both of the sheath and catheter may include a spine comprised of discrete sections, and that the number of discrete sections may vary. Further, a spine of a sheath and/or catheter may be a single element or multi-element or multi-layer spine.

Also, a given discrete section may be structurally distinguished from one or more or all of the other discrete sections based on a single structural attribute or a combination of two, three, four and other numbers of structural attributes. Structural attributes other than the attributes discussed herein may also be utilized. Further, a spine may be formed from a plurality of discrete sections that are attached or pieced together, or the spine may be formed by fabrication methods that process and/or form discrete sections into an integrated or unitary structure.

Additionally, certain system components are described as having lumens that are configured for carrying or passage of control elements, control cables, wires, and other catheter instruments. Such lumens may also be used to deliver fluids such as saline, water, carbon dioxide, nitrogen, helium, for example, in a gaseous or liquid state, to the distal tip. Further, some embodiments may be implemented with a open loop or closed loop cooling system wherein a fluid is passed through one or more lumens in the sidewall of the catheter instrument to cool the catheter or a tool at the distal tip.

Further, embodiments may be utilized with various working instruments including end effectors including, for example, a Kittner dissector, a multi-fire coil tacker, a clip applier, a cautery probe, a shovel cautery instrument, serrated graspers, tethered graspers, helical retraction probe, scalpel, basket capture device, irrigation tool, needle holders, fixation device, transducer, and various other graspers. A number of other catheter type instruments may also be utilized together with certain embodiments including, but not limited to, a mapping catheter, an ablation catheter, an ultrasound catheter, a laser fiber, an illumination fiber, a wire, transmission line, antenna, a dilator, an electrode, a microwave catheter, a cryo-ablation catheter, a balloon catheter, a stent delivery catheter, a fluid/drug delivery tube, a suction tube, an optical fiber, an image capture device, an endoscope, a Foley catheter, Swan-Ganz catheter, fiberscope, etc. Thus, it is contemplated that one or more catheter instruments may be inserted through one or more lumens of a flexible catheter instrument, flexible sheath instrument, or any catheter instrument to reach a surgical site at the distal tip.

Because one or more components of embodiments may be used in minimally invasive surgical procedures, the distal portions of these instruments may not be easily visible to the naked eye. As such, embodiments of the invention may be utilized with various imaging modalities such as magnetic resonance (MR), ultrasound, computer tomography (CT), X-ray, fluoroscopy, etc. may be used to visualize the surgical procedure and progress of these instruments. It may also be desirable to know the precise location of any given catheter instrument and/or tool device at any given moment to avoid undesirable contacts or movements. Thus, embodiments may be utilized with localization techniques that are presently available may be applied to any of the apparatuses and methods disclosed above. A plurality of sensors, including those for sensing patient vitals, temperature, pressure, fluid flow, force, etc., may be combined with the various embodiments of flexible catheters and distal orientation platforms.

Accordingly, embodiments are intended to cover alternatives, modifications, and equivalents that may fall within the scope of the claims.

1. A spine apparatus for a flexible, elongate instrument, comprising:
an elongate body having a proximal end and a distal end, and defining a lumen that extends there between, the elongate body having a plurality of apertures in a wall thereof,
the elongate body comprising a unitary structure having a plurality of discrete sections, each section having at least one distinguishing structural attribute that differentiates it from the other sections,
wherein a distinguishing attribute of at least one of the sections is related to the plurality of apertures, and
wherein a flexibility of the elongate body varies along its length based on the arrangement of the sections.

2. The apparatus of claim 1, wherein a distinguishing structural attribute of multiple sections is related to the plurality of apertures.

3. The apparatus of claim 1, wherein a distinguishing structural attribute related to the plurality of apertures comprises a size of apertures defined by a discrete section.

4. The apparatus of claim 1, wherein a first discrete section defines apertures having a first size, and a second discrete section defines apertures having a second size smaller than the first size.

5. The apparatus of claim 1, wherein the elongate body has a first discrete section which defines apertures that are longer and wider than apertures defined by a second discrete section proximal to the first discrete section, and wherein apertures defined by the second discrete section are longer and wider than apertures defined by a third discrete section proximal to the second discrete section.

6. The apparatus of claim 1, wherein a distinguishing structural attribute related to the plurality of apertures comprises a number of apertures defined by a discrete section.

7. The apparatus of claim 1, wherein a first discrete section defines a first number of apertures, and a second discrete section defines a second number of apertures different from the first number of apertures.

8. The apparatus of claim 7, wherein the apertures defined by the first discrete section are larger than the apertures defined by the second discrete section.

9. The apparatus of claim 1, wherein a distinguishing structural attribute related to the plurality of apertures comprises spacing between the apertures.

10. The apparatus of claim 1, wherein the distinguishing structural attribute related to the plurality of apertures comprises a shape of the apertures.

11. The apparatus of claim 1, wherein a first discrete section defines apertures having a first shape, and a second discrete section defines apertures having a second shape different from the first shape.

12. The apparatus of claim 1, wherein a distinguishing structural attribute related to the plurality of apertures comprises a shape of a middle portion of the respective apertures.

13. The apparatus of claim 12, wherein the middle portion of the respective apertures are I-shaped.

14. The apparatus of claim 13, wherein the I-shaped apertures comprise an enlarged or bulbous middle portion, respective intermediate portions extending from respective ends of the middle portion, and respective end portions extending from the respective intermediate portions, and wherein the intermediate portions are the narrowest portions of the I-shaped apertures.

15. The apparatus of claim 14, wherein the respective middle and end portions have approximately same widths.

16. The apparatus of claim 1, wherein a distinguishing structural attribute related to the plurality of apertures comprises a length of the respective apertures and a degree to which the apertures subtend a respective discrete section.

17. The apparatus of claim 1, wherein a distinguishing structural attribute related to the plurality of apertures comprises the existence of apertures in a respective section.

18. The apparatus of claim 1, wherein a distinguishing structural attribute related to the plurality of apertures comprises a degree to which apertures within a discrete section overlap each other.

19. The apparatus of claim 1, wherein the plurality of apertures have a symmetrical shape or formation.

20. The apparatus of claim 19, wherein each discrete section has a plurality of apertures having a symmetrical shape or formation, wherein the respective apertures of a first discrete section are longer and wider than the respective apertures of a second discrete section proximal of the first discrete section.

21. The apparatus of claim 20, wherein the respective apertures of the second discrete section are longer and wider than the respective apertures of a third discrete section.

22. The apparatus of claim 1, at least two of the discrete sections having a substantially same length.

23. The apparatus of claim 1, wherein the flexible, elongate instrument has a shape and size configured for use in a robotic medical instrument system.

24. A spine apparatus for a flexible, elongate instrument, comprising:
an elongate body having a proximal end and a distal end, and defining a lumen that extends there between,
the elongate body comprising a unitary structure having a plurality of discrete sections, each discrete section having at least one distinguishing structural attribute that differentiates it from the other sections, wherein a flexibility of the elongate body varies along its length based on the arrangement of the sections.

25. The apparatus of claim 24, at least two of the discrete sections having a substantially same length.

26. The apparatus of claim 24, wherein the structural attribute comprises a type of material.

27. The apparatus of claim 24, wherein the structural attribute comprises a dimension of the respective sections.

28. The apparatus of claim 27, wherein the dimension is a wall thickness.

29. The apparatus of claim 27, wherein the dimension is a width or a diameter.

30. The apparatus of claim 29, wherein a discrete section tapers along its length from a first width to a second width that is less than the first width.

31. The apparatus of claim 24, wherein the respective sections are made of a same material, at least one section having a different geometric structure than the other sections, and at least one section having a smaller diameter than the other sections.

32. The apparatus of claim 24, wherein a structural attribute comprises a density of a braid material used in the elongate body.

33. The apparatus of claim 24, wherein the flexible, elongate instrument has a shape and size configured for use in a robotic medical instrument system.

34. A surgical instrument system, comprising:
a flexible, elongate sheath instrument having a sheath body and a plurality of respective control elements that extend through respective lumens defined by the sheath body; and
a catheter instrument coaxially positioned within a central lumen defined by the sheath instrument, the catheter instrument having a catheter body and a plurality of respective control elements that extend through respective lumens defined by the catheter body,
at least one of the sheath and catheter instruments comprising a flexible spine, the spine comprising an elongate body having a proximal end, a distal end and a lumen that extends there between, the elongate body comprising a unitary structure that includes a plurality of discrete sections, each section having at least one structurally distinguishing attribute, such that the flexibilities of the spine and the respective sheath and/or catheter instrument vary along their respective lengths based on the arrangement of the sections.

35. The system of claim 34, the sheath instrument comprising a first spine, and the catheter instrument comprising a second spine, each of the first and second spines comprising an elongate body having a proximal end and a distal end and defining a central lumen that extends there between, the elongate body comprising a unitary structure that includes a plurality of discrete sections, each section having at least one distinguishing attribute that structurally differentiates it from the other respective sections of the respective spine.

36. The system of claim 35, wherein the first and second spines have a substantially same shape, and wherein the first spine is larger than the second spine.

37-50. (canceled)
